(19) United States
(12) Patent Application Publication
(10) Pub. No.: US 2025/0301906 A1
(43) Pub. Date: Sep. 25, 2025

(54) CARBAZOLE-BASED NON-FULLERENES ELECTRON ACCEPTORS AND COMPOSITIONS THEREOF USEFUL AS ORGANOVOLTAIC MATERIAL

(71) Applicant: SOCPRA SCIENCES ET GÉNIE S.E.C., Sherbrooke (CA)

(72) Inventor: Pierre HARVEY, Sherbrooke (CA)

(73) Assignee: SOCPRA SCIENCES ET GÉNIE S.E.C., Sherbrooke, QC (CA)

(21) Appl. No.: 19/105,067

(22) PCT Filed: Aug. 29, 2023
(86) PCT No.: PCT/CA2023/051142

§ 371 Date: Feb. 20, 2025

Related U.S. Application Data
(60) Provisional application No. 63/373,785, filed on Aug. 29, 2022.

(51) Int. Cl. H10K 85/60; C07D 209/86; H10K 30/30; H10K 30/50; H10K 30/82

(52) U.S. Cl. CPC H10K 85/6572; C07D 209/86; H10K 30/30; H10K 30/50; H10K 30/82

(57) ABSTRACT

Compounds of Formula Ia and Ib: (Ia) (Ib) wherein R, R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN, are disclosed herein. Organic photovoltaics (OPVs) based on binary and ternary compositions comprising a compound of Formula Ia and/or Ib are also disclosed. In an aspect, a compound of Formula Ia (electron acceptor material A₁) was combined with PTB7-Th (electron donor material D₁) to afford binary bulk heterojunction blends. In an aspect, a pair of compounds of Formula Ia (electron acceptor materials A₁) were combined with PTB7-Th (electron donor material D₁) to afford ternary bulk heterojunction blends. In a further aspect, a compound of Formula Ib (electron acceptor material A₁) was combined with P3HT (electron donor material D₁) to afford binary bulk heterojunction blends.

### CROSS REFERENCE TO RELATED APPLICATIONS

This application claims the benefit of U.S. Provisional Application 63/373,785, filed Aug. 29, 2022. The contents of the referenced application are incorporated into the present application by reference.

### FIELD

The present disclosure broadly relates to novel organovoltaic materials and uses thereof. More specifically but not exclusively, the present disclosure relates to novel blends for low intensity indoor light harvesting. Yet more specifically but not exclusively, the present disclosure relates to novel organovoltaic materials based on binary or ternary bulk heterojunction (BHJ) blends. The present disclosure also relates to a process for the preparation of novel organovoltaic materials based on binary or ternary bulk heterojunction (BHJ) blends. Moreover, the present disclosure relates to the use of the novel organovoltaic materials in organic electronics.

### BACKGROUND

Indoor photovoltaics (iPVs) target the harvesting of artificial light and are expected to play a vital role as a power supply source for Internet of Things (IoT) systems. One immediate market entry point for iPVs is powering small electronic devices, as tens of billions of these devices are expected to be installed within the coming decade. Indoor light intensities in the range of 500 lux (office spaces) and 1000 lux (factories) are sufficient to provide >100 μW power when using small iPVs modules. This power is enough to supply smart IoT devices such as radio frequency identification (RFID) tags (˜10 μW), ecobee thermostats (˜18 μW), and passive WiFi (˜60 μW). Critical to iPV deployment are the following: i) bandgap engineering of the active layer—employing photoactive materials with medium bandgaps (visible range light absorption) to match the emission of LED lighting; ii) high V_{oc} values to offset the voltage loss under low-light intensity;^{[1]} and iii) vetting device performance at scale.

The incorporation of non-fullerenes as electron acceptors in organic solar cells (OSC) has greatly contributed to increasing the power conversion efficiency (PCE), in certain cases by up to ˜30%, by capturing more solar energy. The general design of these non-fullerene containing materials typically comprises a conjugated A-D-A structure. Carbazole and indolocarbazole based non-fullerenes have been explored.^{[2, 3]} The short term goal in structure design is to reach and exceed PCEs of 20%.^{[4]} Recent studies reported PCEs of 17.1%^{[5, 6]}, 17.2%^{[7, 8]}, 17.48%^{[9]}, 17.6%^{[10, 11]}, 17.7%^{[12]}, 18.01%^{[13]}, 18.16%^{[14]}, and 18.38% at AM (Air Mass) 1.5^{[15]}. Moreover, a PCE of 20.73% was recently reported for a carbazole-based non-fullerene under 1000 lux and 3000 K.^{[16]}

Carbazole-based D-A non-fullerenes have also been examined, but the reported PCEs were generally modest (FIG. 1).^{[17-19]} To that effect, (D-A)1 has been shown to provide the best performance, with a PCE of 9.29%, which is rather surprising in view of N-substituted carbazole (D-A) derivatives (D=N-phenylcarbazole; A=2-(3-oxo-2,3-dihydro-1H-inden-1-ylidene)malononitrile and 2-(1,3-dihydro-1,3-dioxo-2H-inden-2-ylidene) exhibiting rather low electronic field induced 2nd harmonic (EFISH) p values.^{[20]} This implies that the charge transfer process is not as extensive rendering these molecules unsuitable as non-fullerene acceptors.

Novel carbazole-based non-fullerene electron acceptors providing for improved PCE values are of commercial interest. Moreover, binary and ternary blends comprising such novel carbazole-based non-fullerene electron acceptors, and devices based on such blends, are of commercial interest.

### SUMMARY

The present disclosure broadly relates to novel organovoltaic materials and uses thereof. More specifically but not exclusively, the present disclosure relates to novel blends for low intensity indoor light harvesting. Yet more specifically but not exclusively, the present disclosure relates to novel organovoltaic materials based on binary or ternary bulk heterojunction (BHJ) blends. The present disclosure also relates to a process for the preparation of novel organovoltaic materials based on binary or ternary bulk heterojunction (BHJ) blends. Moreover, the present disclosure relates to the use of the novel organovoltaic materials in organic electronics.

A solution to the problems associated with the development of novel and cost-efficient compounds suitable for use in OPV devices has been discovered. Broadly, the solution resides in the discovery of novel carbazole-based non-fullerenes suitable for use in OPV devices. In an aspect, the present disclosure relates to a binary blend comprising an electron donor material and an electron acceptor material. In a further aspect, the present disclosure relates to a ternary blend comprising an electron donor material and at least two electron acceptor materials. In a further aspect, the present disclosure relates to a binary blend comprising an electron donor material and a carbazole-based non-fullerene electron acceptor material. In a further aspect, the present disclosure relates to a ternary blend comprising an electron donor material and a first and second carbazole-based non-fullerene electron acceptor material. These binary and ternary blends may be advantageously used in OPVs for efficient indoor light harvesting.

In an aspect, the present disclosure relates to a compound of Formula Ia:

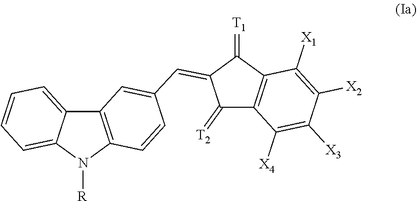

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a compound of Formula Ib:

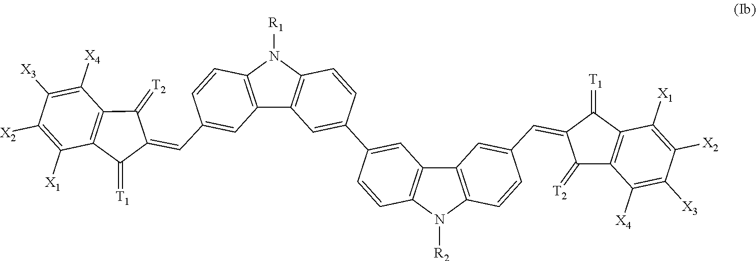

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a composition comprising an electron acceptor material A₁ and an electron donor material D₁, wherein the electron acceptor material A₁ is of Formula Ia:

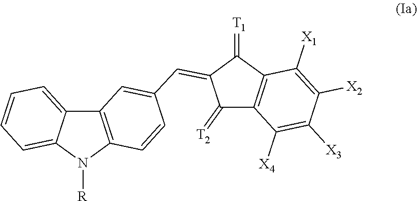

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a composition comprising an electron acceptor material A₁ and an electron donor material D₁, wherein the electron acceptor material A₁ is of Formula Ib:

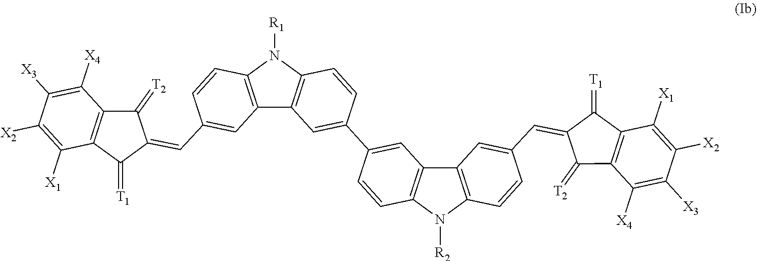

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a composition comprising at least two electron acceptor materials A₁ and A₂, and at least an electron donor material D₁, wherein the electron acceptor materials A₁ and A₂, are of Formula Ia:

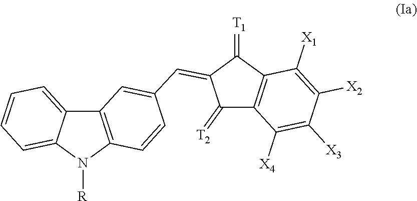

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a composition comprising at least two electron acceptor materials A₁ and A₂, and at least an electron donor material D₁, wherein the electron acceptor materials A₁ and A₂, are of Formula Ib:

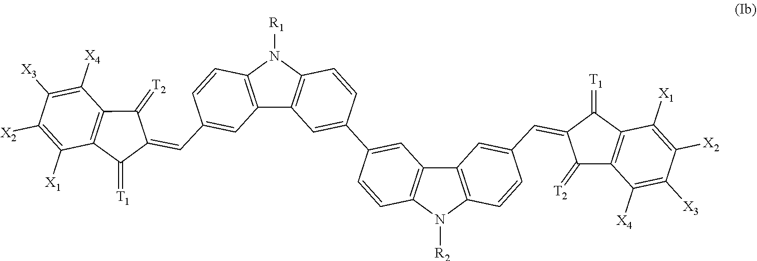

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a composition comprising at least two electron acceptor materials A₁ and A₂, and at least an electron donor material D₁, wherein the electron acceptor materials A₁ and A₂, are of Formula Ia and/or Ib:

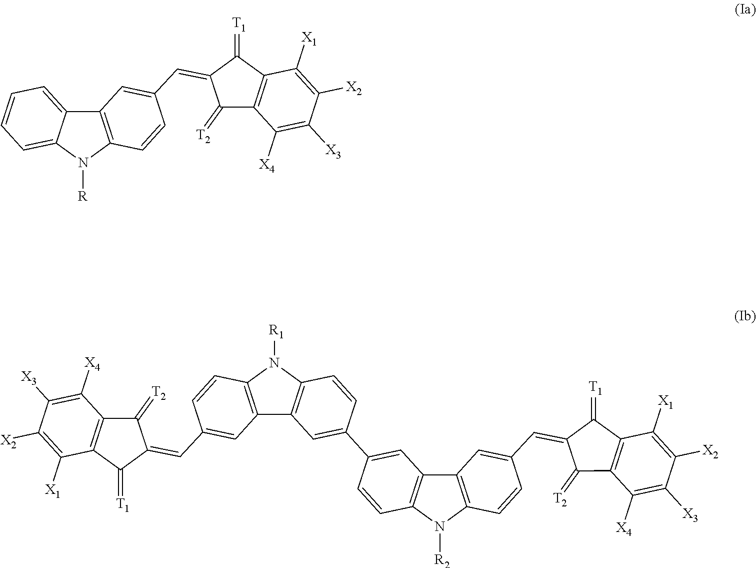

wherein R, R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an embodiment of the present disclosure, the mass ratio (w/w) of the acceptor material(s) and the donor material may be from about 0.5:1.0 to about 1.0:0.5, for example from about 0.6:0.9 to about 0.9:0.6, for example from about 0.7:0.8 to about 0.8:0.7, or any range derivable therein, for example about 0.75:0.75. In a particular embodiment, the binary blend comprises an acceptor:donor ratio (w/w) from about 0.5:1.0 to about 1.0:0.5. In a particular embodiment, the ternary blend comprises an acceptor:donor ratio (w/w) from about 0.5:1.0 to about 1.0:0.5. In a further embodiment of the present disclosure, the binary and ternary blends may be slot-die coated from environmentally friendly solvents (e.g., halogen-free solvents).

In an aspect, the present disclosure relates to binary compositions exhibiting band gaps sufficiently matching the full indoor LED emission spectrum, the compositions comprising an electron acceptor material A₁ and an electron donor material D₁, wherein the electron acceptor material A₁ is of Formula Ia:

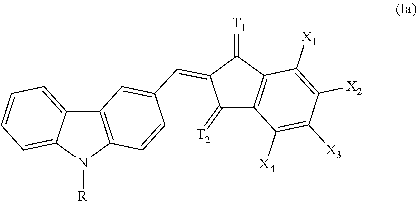

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to binary compositions exhibiting band gaps sufficiently matching the full indoor LED emission spectrum, the compositions comprising an electron acceptor material A₁ and an electron donor material D₁, wherein the electron acceptor material A₁ is of Formula Ib:

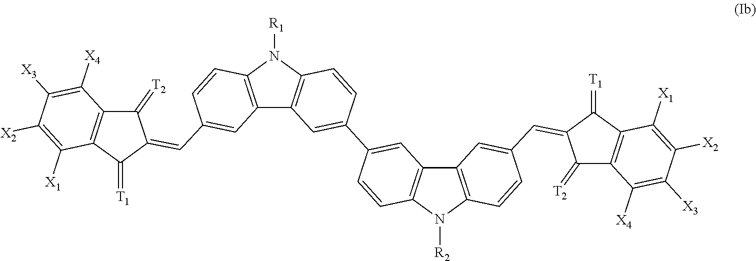

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to ternary compositions exhibiting band gaps sufficiently matching the full indoor LED emission spectrum, the compositions comprising at least two electron acceptor materials A₁ and A₂, and at least an electron donor material D₁, wherein the electron acceptor materials A₁ and A₂, are of Formula Ia:

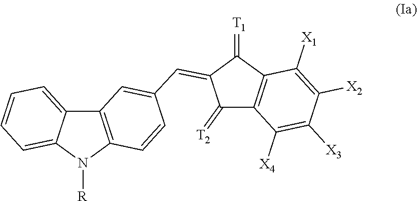

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to ternary compositions exhibiting band gaps sufficiently matching the full indoor LED emission spectrum, the compositions comprising at least two electron acceptor materials A₁ and A₂, and at least an electron donor material D₁, wherein the electron acceptor materials A₁ and A₂, are of Formula Ib:

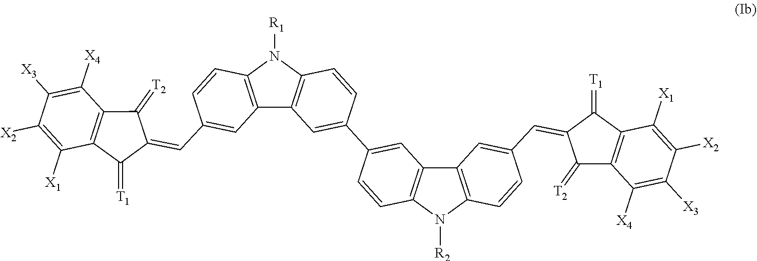

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to ternary compositions exhibiting band gaps sufficiently matching the full indoor LED emission spectrum, the compositions comprising at least two electron acceptor materials A₁ and A₂, and at least an electron donor material D₁, wherein the electron acceptor materials A₁ and A₂, are of Formula Ia and/or Ib:

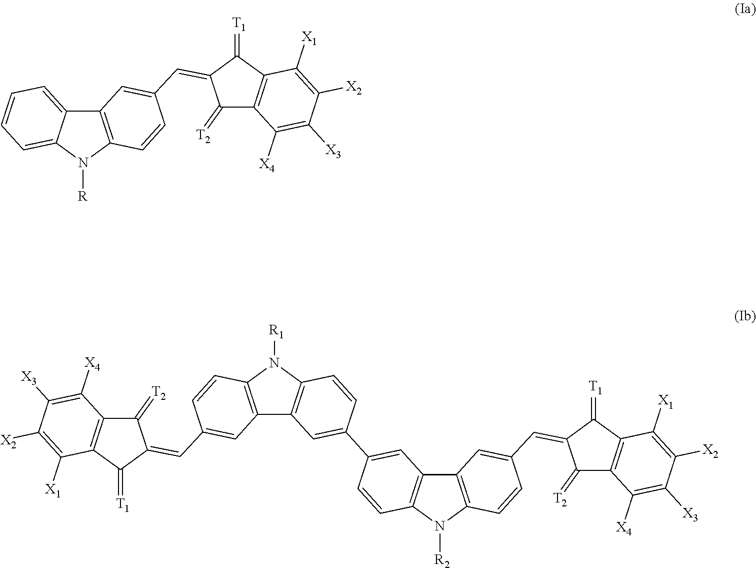

wherein R, R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an aspect, the present disclosure relates to a binary composition comprising an electron acceptor material A₁ and an electron donor material D₁, wherein the electron acceptor material A₁ is of Formula Ia:

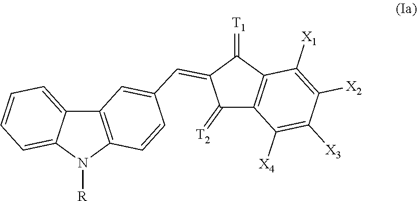

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})).

In an aspect, the present disclosure relates to a ternary composition comprising two electron acceptor materials A₁ and A₂, and at least an electron donor material D₁, wherein the electron acceptor materials A₁ and A₂, are of Formula Ia:

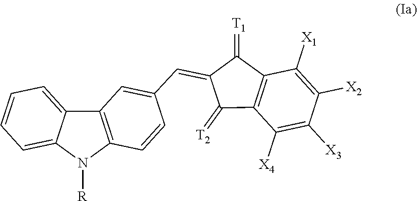

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})).

In an aspect, the present disclosure relates to a binary composition comprising an electron acceptor material A₁ and an electron donor material D₁, wherein the electron acceptor material A₁ is of Formula Ib:

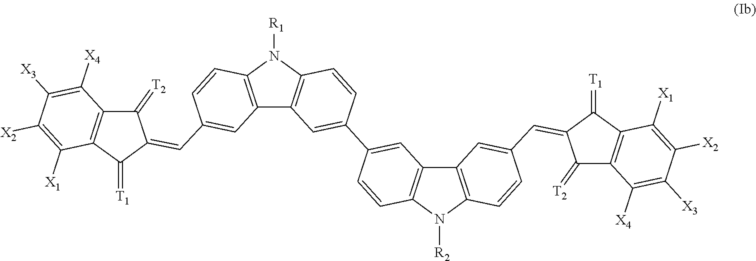

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT).

In an aspect, the present disclosure relates to a ternary composition comprising two electron acceptor materials A₁ and A₂, and at least an electron donor material D₁, wherein the electron acceptor materials A₁ and A₂, are of Formula Ib:

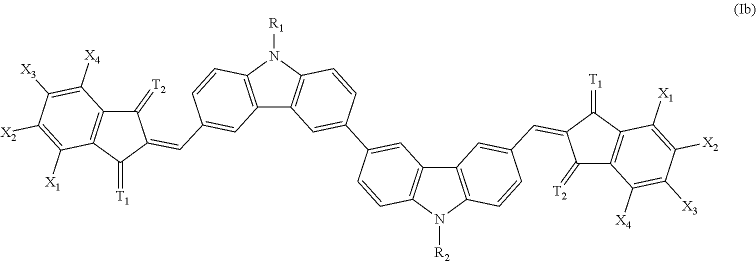

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT).

In an aspect, the present disclosure relates to a photoactive layer for iOPV (indoor organic photovoltaics) construction having high thermal stability and strong visible light absorption. In an embodiment, the photoactive layer comprises a binary composition comprising an electron acceptor material A₁ and an electron donor material D₁, wherein the electron acceptor material A₁ is of Formula Ia:

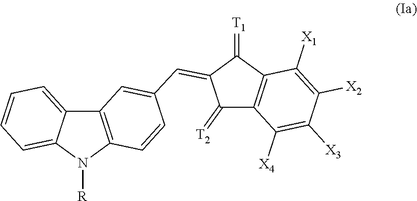

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})).

In an aspect, the present disclosure relates to a photoactive layer for iOPV (indoor organic photovoltaics) construction having high thermal stability and strong visible light absorption. In an embodiment, the photoactive layer comprises a ternary composition comprising two electron acceptor materials A₁ and A₂, and at least an electron donor material D₁, wherein the electron acceptor materials A₁ and A₂, are of Formula Ia:

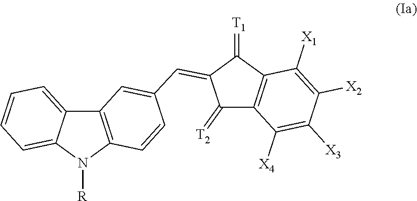

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})).

In an aspect, the present disclosure relates to a photoactive layer for iOPV (indoor organic photovoltaics) construction having high thermal stability and strong visible light absorption. In an embodiment, the photoactive layer comprises a binary composition comprising an electron acceptor material A₁ and an electron donor material D₁, wherein the electron acceptor material A₁ is of Formula Ib:

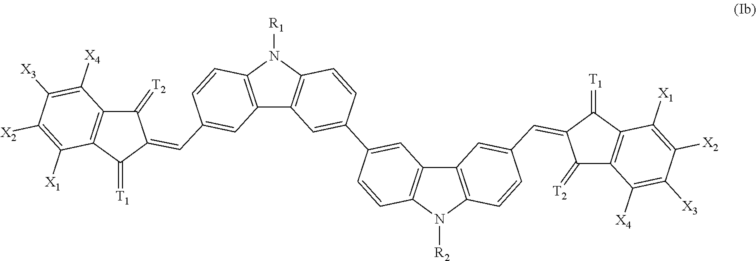

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT).

In an aspect, the present disclosure relates to a photoactive layer for iOPV (indoor organic photovoltaics) construction having high thermal stability and strong visible light absorption. In an embodiment, the photoactive layer comprises a ternary composition comprising two electron acceptor materials A₁ and A₂, and at least an electron donor material D₁, wherein the electron acceptor materials A₁ and A₂, are of Formula Ib:

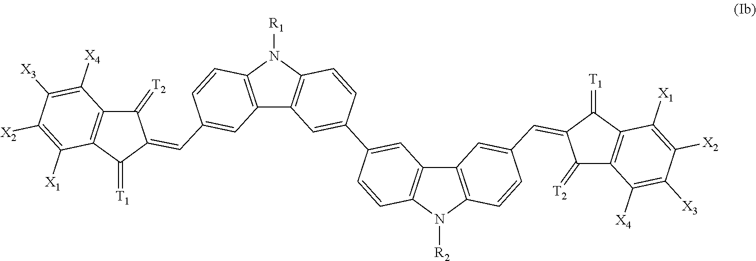

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT).

In an aspect, the present disclosure relates to the use of a binary composition comprising an electron acceptor material A₁ and an electron donor material D₁, wherein the electron acceptor material A₁ is of Formula Ia:

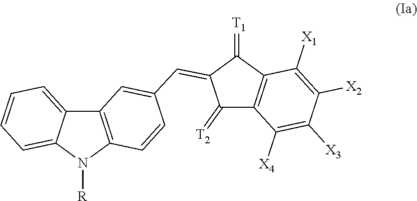

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})), for enhancing device photocurrent generation. In an embodiment of the present disclosure the device is an OPV used for indoor light recycling.

In an aspect, the present disclosure relates to the use of a ternary composition comprising two electron acceptor materials A₁ and A₂, and at least an electron donor material D₁, wherein the electron acceptor materials A₁ and A₂, are of Formula Ia:

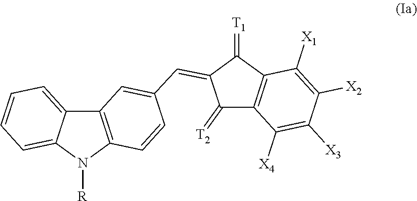

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})), for enhancing device photocurrent generation. In an embodiment of the present disclosure the device is an OPV used for indoor light recycling.

In an aspect, the present disclosure relates to the use of a binary composition comprising an electron acceptor material A₁ and an electron donor material D₁, wherein the electron acceptor material A₁ is of Formula Ib:

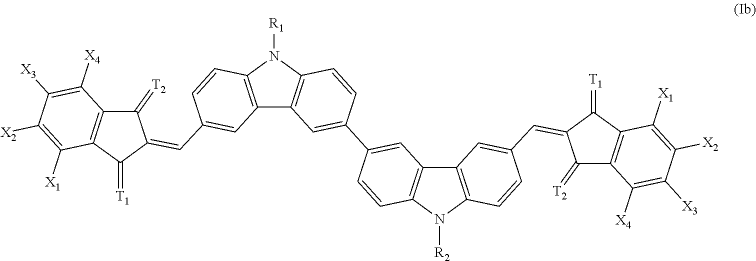

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT), for enhancing device photocurrent generation. In an embodiment of the present disclosure the device is an OPV used for indoor light recycling.

In an aspect, the present disclosure relates to the use of a ternary composition comprising two electron acceptor materials A₁ and A₂, and at least an electron donor material D₁, wherein the electron acceptor materials A₁ and A₂, are of Formula Ib:

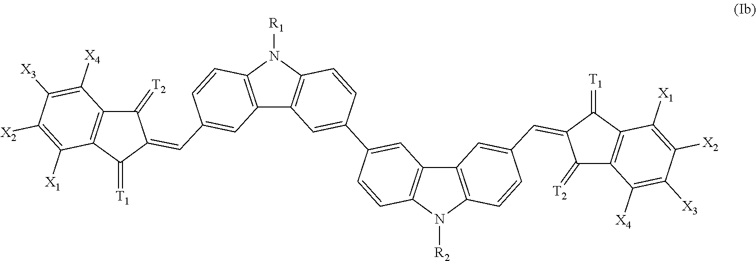

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT), for enhancing device photocurrent generation. In an embodiment of the present disclosure the device is an OPV used for indoor light recycling.

In an aspect, the present disclosure relates to the use of a binary composition comprising an electron acceptor material A₁ and an electron donor material D₁, wherein the electron acceptor material A₁ is of Formula Ia:

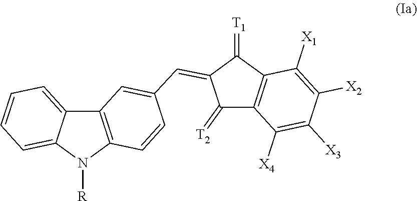

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})), as an organic semiconductor material, layer or component.

In an aspect, the present disclosure relates to the use of a ternary composition comprising two electron acceptor materials A₁ and A₂, and at least an electron donor material D₁, wherein the electron acceptor materials A₁ and A₂, are of Formula Ia:

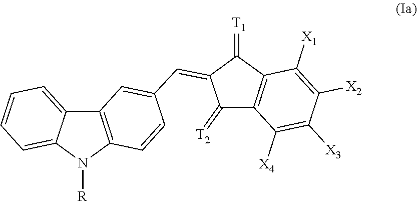

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})), as an organic semiconductor material, layer or component.

In an aspect, the present disclosure relates to the use of a binary composition comprising an electron acceptor material A₁ and an electron donor material D₁, wherein the electron acceptor material A₁ is of Formula Ib:

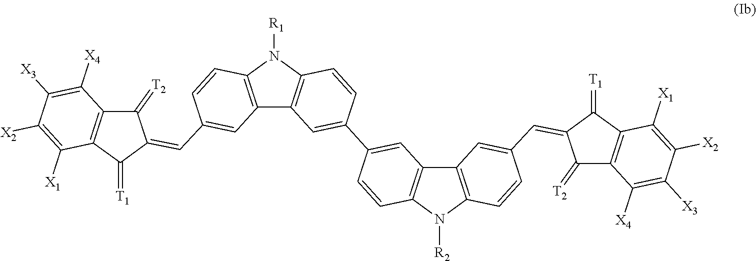

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT), as an organic semiconductor material, layer or component.

In an aspect, the present disclosure relates to the use of a ternary composition comprising two electron acceptor materials A₁ and A₂, and at least an electron donor material D₁, wherein the electron acceptor materials A₁ and A₂, are of Formula Ib:

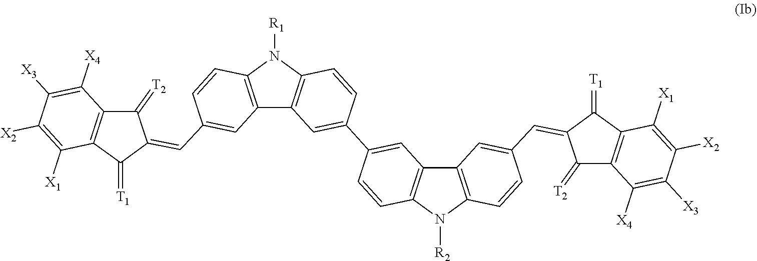

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT), as an organic semiconductor material, layer or component.

In an aspect, the present disclosure relates to an organic solar cell, comprising: an anode and a cathode; and a photoactive layer between the anode and the cathode; wherein the photoactive layer comprises an electron donor/acceptor material, wherein the electron acceptor material is of Formula Ia:

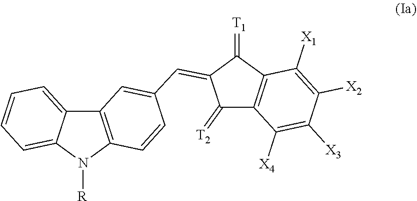

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})).

In an aspect, the present disclosure relates to an organic solar cell, comprising: an anode and a cathode; and a photoactive layer between the anode and the cathode; wherein the photoactive layer comprises an electron donor/acceptor material, wherein the electron acceptor material is of Formula Ib:

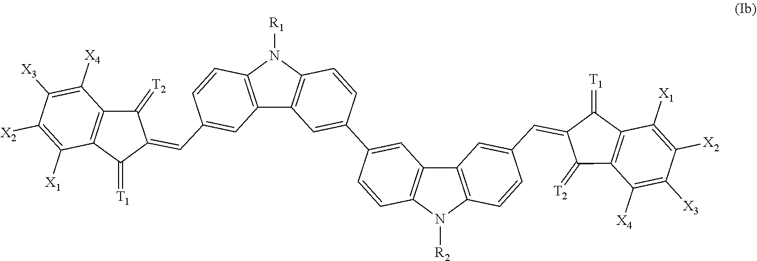

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT).

In an aspect, the present disclosure relates to an electronic device comprising a heterojunction, wherein the heterojunction comprises a composition comprising an electron donor and an electron acceptor material, wherein the electron acceptor material is of Formula Ia:

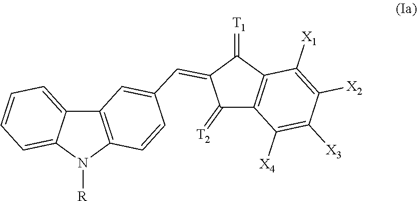

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})). In an embodiment of the present disclosure, the electronic device is an OPV device. In a further embodiment of the present disclosure, the OPV is used for indoor light harvesting.

In an aspect, the present disclosure relates to an electronic device comprising a heterojunction, wherein the heterojunction comprises a composition comprising two electron acceptor materials A₁ and A₂, and at least an electron donor material D₁, wherein the electron acceptor materials A₁ and A₂, are of Formula Ia:

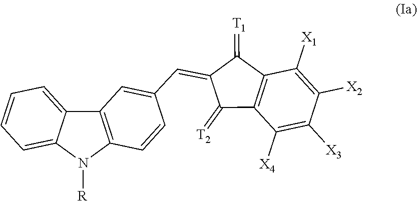

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})). In an embodiment of the present disclosure, the electronic device is an OPV device. In a further embodiment of the present disclosure, the OPV is used for indoor light harvesting.

In an aspect, the present disclosure relates to an electronic device comprising a heterojunction, wherein the heterojunction comprises a composition comprising an electron donor and an electron acceptor material, wherein the electron acceptor material is of Formula Ib:

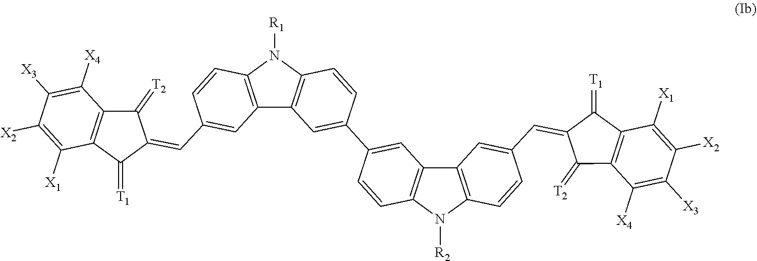

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT). In an embodiment of the present disclosure, the electronic device is an OPV device. In a further embodiment of the present disclosure, the OPV is used for indoor light harvesting.

In an aspect, the present disclosure relates to an electronic device comprising a heterojunction, wherein the heterojunction comprises a composition comprising two electron acceptor materials A₁ and A₂, and at least an electron donor material D₁, wherein the electron acceptor materials A₁ and A₂, are of Formula Ib:

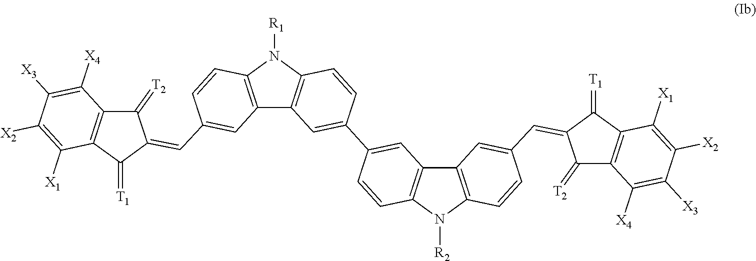

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN; and wherein the electron donor material D₁ may be poly(3-hexylthiophene) (P3HT). In an embodiment of the present disclosure, the electronic device is an OPV device. In a further embodiment of the present disclosure, the OPV is used for indoor light harvesting.

In an aspect, the present disclosure relates to a compound of Formula:

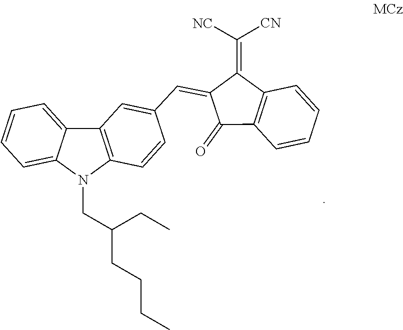 [SMILES: CCCCC(CC)Cn1c2ccccc2c2cc(/C=C3\C(=O)c4ccccc4C3=C(C#N)C#N)ccc21]

In an aspect, the present disclosure relates to a compound of Formula:

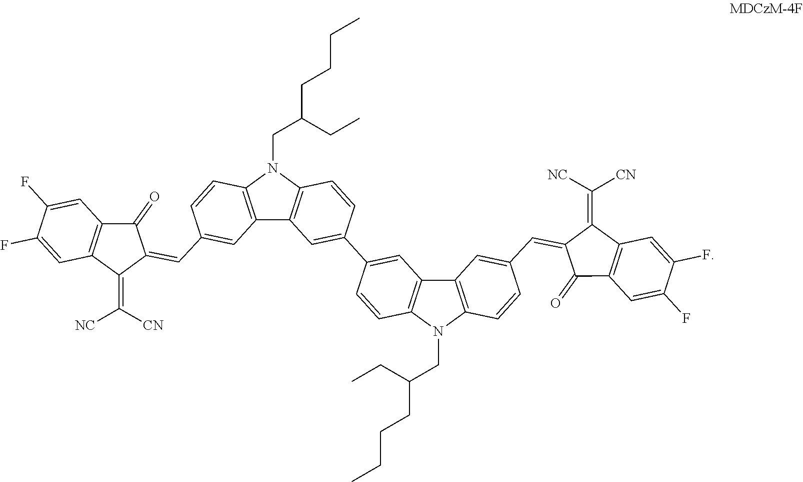 [SMILES: CCCCC(CC)Cn1c2ccc(/C=C3\C(=O)c4cc(F)c(F)cc4C3=C(C#N)C#N)cc2c2cc(-c3ccc4c(c3)c3cc(/C=C5\C(=O)c6cc(F)c(F)cc6C5=C(C#N)C#N)ccc3n4CC(CC)CCCC)ccc21]

In an aspect, the present disclosure relates to a compound of Formula:

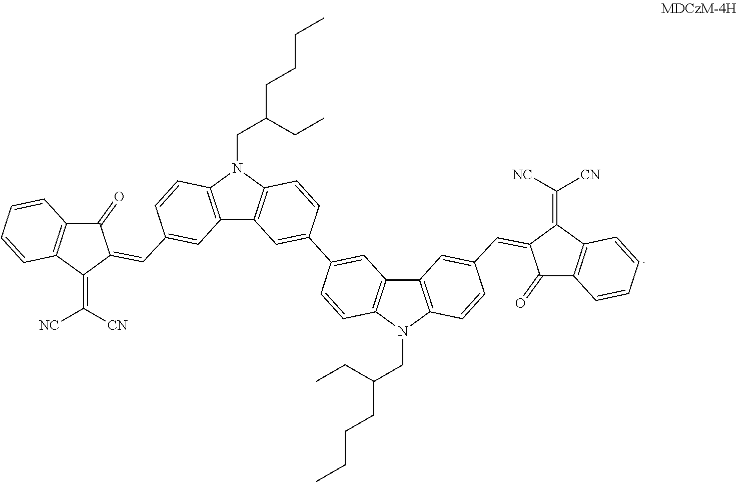 [SMILES: CCCCC(CC)Cn1c2ccc(/C=C3\C(=O)c4ccccc4C3=C(C#N)C#N)cc2c2cc(-c3ccc4c(c3)c3cc(/C=C5\C(=O)c6ccccc6C5=C(C#N)C#N)ccc3n4CC(CC)CCCC)ccc21]

Also disclosed in the context of the present disclosure are embodiments 1 to 93. Embodiment 1 is a compound of Formula Ia:

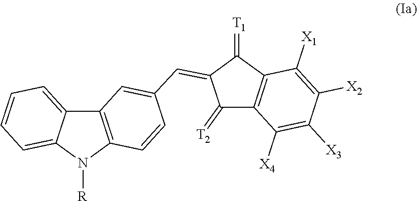

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 2 is the compound of embodiment 1, comprising a structure of Formula Ila:

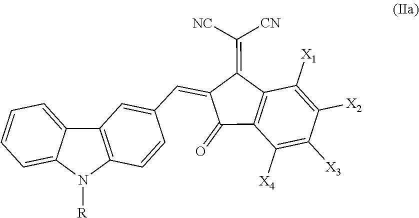

wherein R is a linear or branched C₆₋₁₅-alkyl; and X₁, X₂, X₃ and X₄ are each independently H, F, Cl or Br. Embodiment 3 is the compound of embodiment 1, comprising a structure of Formula IIIa:

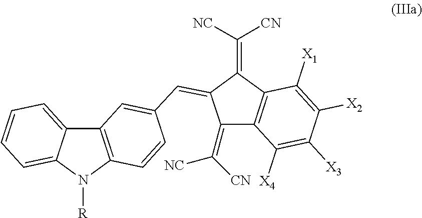

wherein R is a linear or branched C₆₋₁₅-alkyl; and X₁, X₂, X₃ and X₄ are each independently H, F, Cl or Br. Embodiment 4 is the compound of embodiment 1, comprising a structure of Formula IVa:

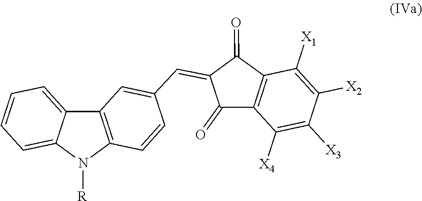

wherein R is a linear or branched C₆₋₁₅-alkyl; and X₁, X₂, X₃ and X₄ are each independently H, F, Cl or Br. Embodiment 5 is the compound of any one of embodiments 2 to 4, wherein X₁, X₂, X₃ and X₄ are each independently H. Embodiment 6 is the compound of embodiment 5, wherein R is 2-ethylhexyl.

Embodiment 7 is a compound of Formula Ib:

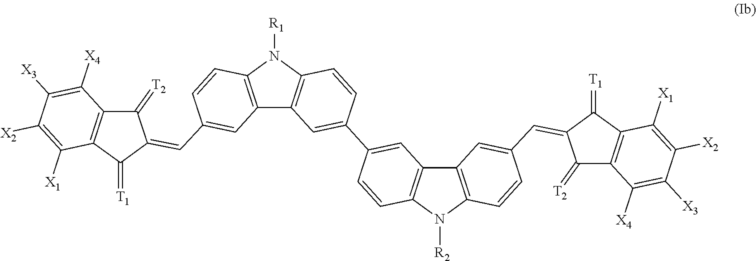

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 8 is the compound of embodiment 7, comprising a structure of Formula IIb:

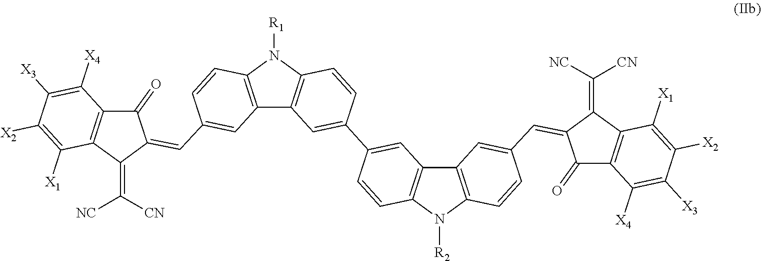

wherein R₁ and R₂ are each independently a linear or branched C₆₋₁₅-alkyl; and X₁, X₂, X₃ and X₄ are each independently H, F, Cl or Br. Embodiment 9 is the compound of embodiment 8, comprising a structure of Formula IIIb:

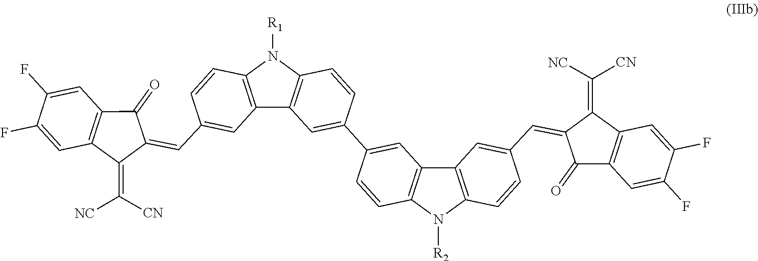

wherein R₁ and R₂ are each independently a linear or branched C₆₋₁₅-alkyl. Embodiment 10 is the compound of embodiment 8, comprising a structure of Formula IVb:

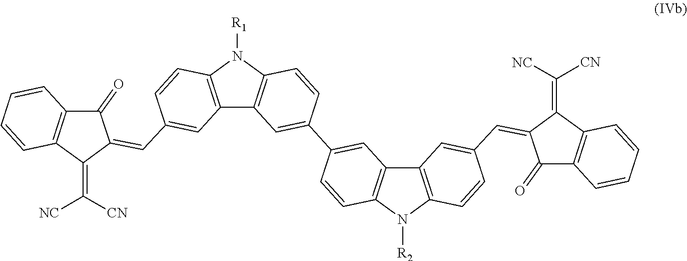

wherein R₁ and R₂ are each independently a linear or branched C₆₋₁₅-alkyl. Embodiment 11 is the compound of any one of embodiments 7 to 10, wherein R is 2-ethylhexyl.

Embodiment 12 is a composition comprising an electron acceptor material A₁ and an electron donor material D₁, wherein the electron acceptor material A₁ is of Formula Ia:

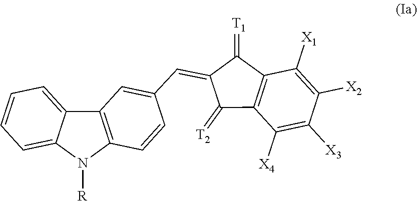

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 13 is the composition of embodiment 12, wherein the electron acceptor material A₁ has the structure of Formula Ila:

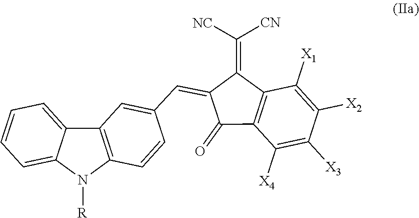

wherein R is a linear or branched C₆₋₁₅-alkyl; and X₁, X₂, X₃ and X₄ are each independently H, F, Cl or Br. Embodiment 14 is the composition of embodiment 12, wherein the electron acceptor material A₁ has the structure of Formula IIIa:

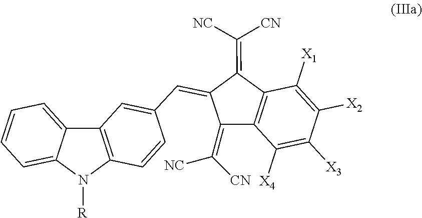

wherein R is a linear or branched C₆₋₁₅-alkyl; and X₁, X₂, X₃ and X₄ are each independently H, F, Cl or Br. Embodiment 15 is the composition of embodiment 12, wherein the electron acceptor material A₁ has the structure of Formula IVa:

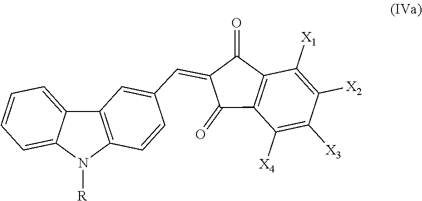

wherein R is a linear or branched C₆₋₁₅-alkyl; and X₁, X₂, X₃ and X₄ are each independently H, F, Cl or Br. Embodiment 16 is the composition of any one of embodiments 12 to 15, wherein X₁, X₂, X₃ and X₄ are each independently H. Embodiment 17 is the composition of embodiment 16, wherein R is 2-ethylhexyl.

Embodiment 18 is a composition comprising an electron acceptor material A₁ and an electron donor material D₁, wherein the electron acceptor material A₁ is of Formula Ib:

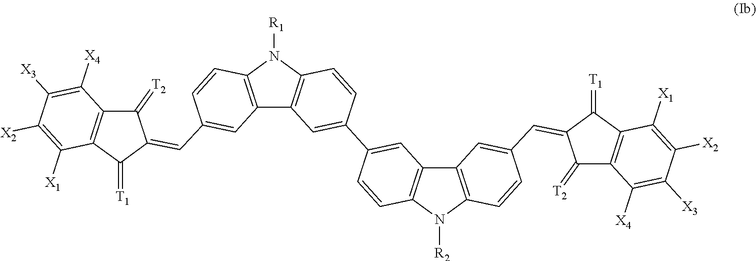

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 19 is the composition of embodiment 18, wherein the electron acceptor material A₁ has the structure of Formula IIb:

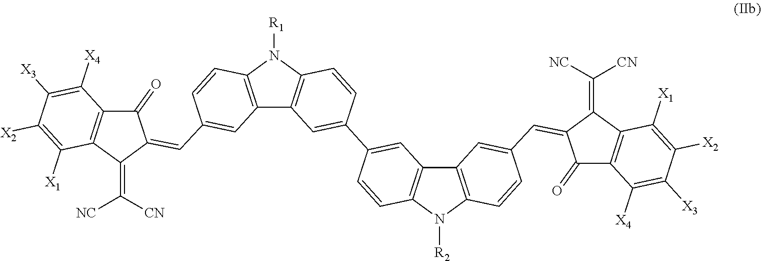

wherein R₁ and R₂ are each independently a linear or branched C₆₋₁₅-alkyl; and X₁, X₂, X₃ and X₄ are each independently H, F, Cl or Br. Embodiment 20 is the composition of embodiment 18, wherein the electron acceptor material A₁ has the structure of Formula IIIb:

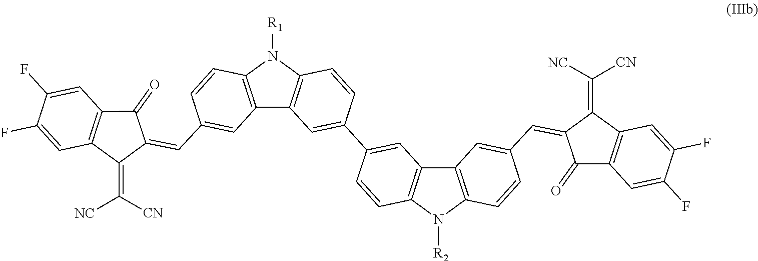

wherein R₁ and R₂ are each independently a linear or branched C₆₋₁₅-alkyl. Embodiment 21 is the composition of embodiment 18, wherein the electron acceptor material A₁ has the structure of Formula IVb:

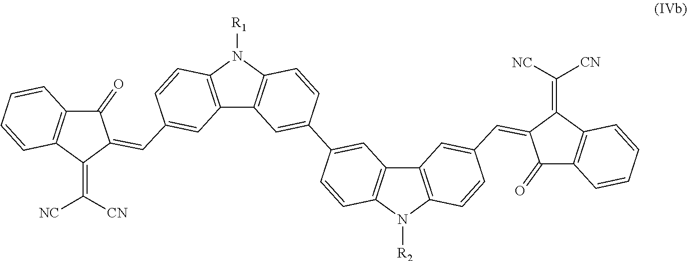

wherein R₁ and R₂ are each independently a linear or branched C₆₋₁₅-alkyl. Embodiment 22 is the composition of any one of embodiments 18 to 21, wherein R is 2-ethylhexyl. Embodiment 23 is the composition of any one of embodiments 12 to 22, wherein the electron donor material D₁ comprises a p-type organic semiconductor material. Embodiment 24 is the composition of any one of embodiments 12 to 23, wherein the electron donor material D₁ comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT). Embodiment 25 is the composition of any one of embodiments 12 or 24, wherein the composition comprises a mass ratio (w/w) of acceptor material to donor material ranging from about 0.5:1.0 to about 1.0:0.5. Embodiment 26 is the composition of any one of embodiments 12 to 25, wherein the composition is slot die coated. Embodiment 27 is the composition of any one of embodiments 12 to 25, wherein the composition is spin coated. Embodiment 28 is the composition of any one of embodiments 12 to 27, wherein the composition produces a band gap suitable for low intensity light harvesting, and wherein the blend has significant absorption of visible light between 380 nm and 940 nm suitable for low intensity light harvesting. Embodiment 29 is the composition of any one of embodiments 12 to 28, wherein the composition is provided in the form of a bulk material or a film.

Embodiment 30 is a composition comprising at least two electron acceptor materials A₁ and A₂, and at least an electron donor material D₁, wherein the electron acceptor materials A₁ and A₂, are of Formula Ia:

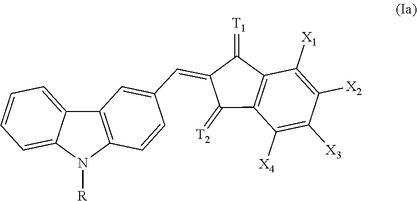

wherein R is a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 31 is the composition of embodiment 30, wherein at least one of the two electron acceptor materials A₁ and A₂ has the structure of Formula Ila:

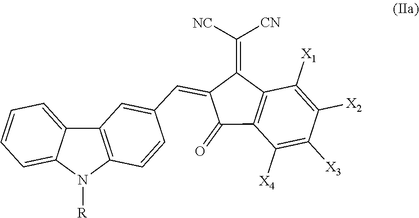

wherein R is a linear or branched C₆₋₁₅-alkyl; and X₁, X₂, X₃ and X₄ are each independently H, F, Cl or Br. Embodiment 32 is the composition of embodiment 30, wherein at least one of the two electron acceptor materials A₁ and A₂ has the structure of Formula IIIa:

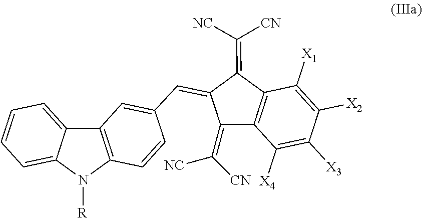

wherein R is a linear or branched C₆₋₁₅-alkyl; and X₁, X₂, X₃ and X₄ are each independently H, F, Cl or Br. Embodiment 33 is the composition of embodiment 30, wherein at least one of the two electron acceptor materials A₁ and A₂ has the structure of Formula IVa:

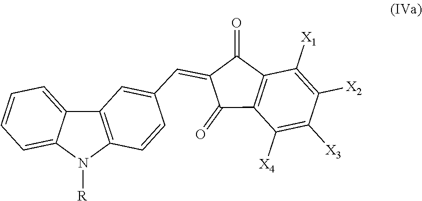

wherein R is a linear or branched C₆₋₁₅-alkyl; and X₁, X₂, X₃ and X₄ are each independently H, F, Cl or Br. Embodiment 34 is the composition of any one of embodiments 30 to 33, wherein X₁, X₂, X₃ and X₄ are each independently H. Embodiment 35 is the composition of embodiment 34, wherein R is 2-ethylhexyl.

Embodiment 36 is a composition comprising at least two electron acceptor materials A₁ and A₂, and at least an electron donor material D₁, wherein the electron acceptor materials A₁ and A₂, are of Formula Ib:

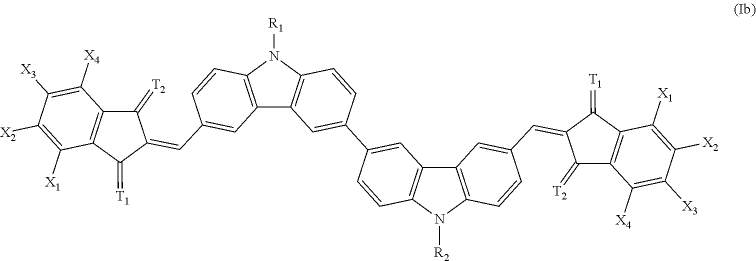

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 37 is the composition of embodiment 36, wherein at least one of the two electron acceptor materials A₁ and A₂ has the structure of Formula IIb:

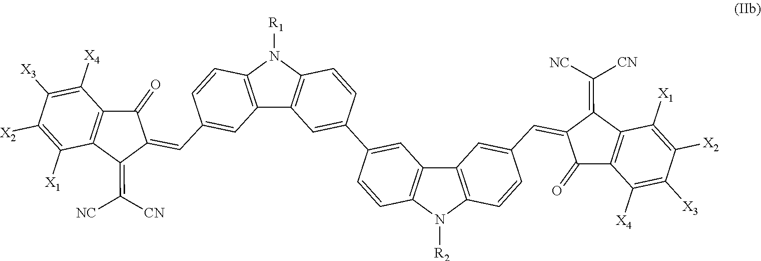

wherein R₁ and R₂ are each independently a linear or branched C₆₋₁₅-alkyl; and X₁, X₂, X₃ and X₄ are each independently H, F, Cl or Br. Embodiment 38 is the composition of embodiment 36, wherein at least one of the two electron acceptor materials A₁ and A₂ has the structure of Formula IIIb:

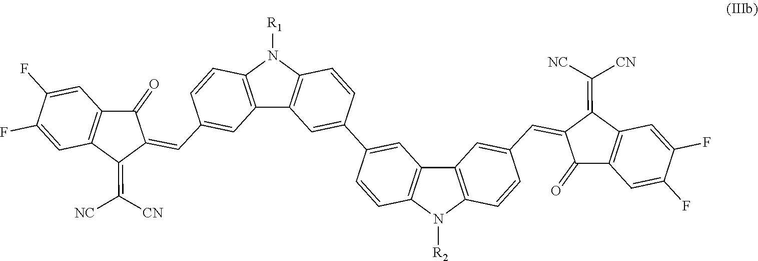

wherein R₁ and R₂ are each independently a linear or branched C₆₋₁₅-alkyl. Embodiment 39 is the composition of embodiment 36, wherein at least one of the two electron acceptor materials A₁ and A₂ has the structure of Formula IVb:

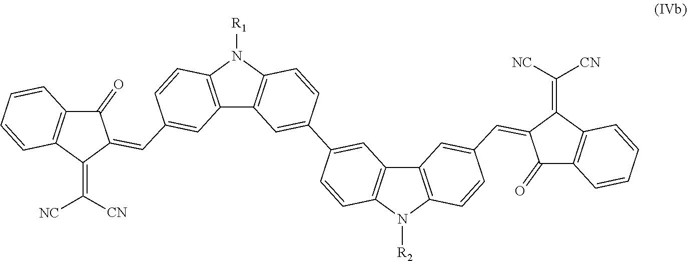

wherein R₁ and R₂ are each independently a linear or branched C₆₋₁₅-alkyl. Embodiment 40 is the composition of any one of embodiments 36 to 39, wherein R is 2-ethylhexyl. Embodiment 41 is the composition of any one of embodiments 30 to 40, wherein the electron donor material D₁ comprises a p-type organic semiconductor material. Embodiment 42 is the composition of any one of embodiments 30 to 41, wherein the electron donor material D₁ comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT). Embodiment 43 is the composition of any one of embodiments 30 to 42, wherein the composition comprises a mass ratio (w/w) of acceptor material to donor material ranging from about 0.5:1.0 to about 1.0:0.5. Embodiment 44 is the composition of any one of embodiments 30 to 43, wherein the composition is slot die coated. Embodiment 45 is the composition of any one of embodiments 30 to 43, wherein the composition is spin coated. Embodiment 46 is the composition of any one of embodiments 30 to 45, wherein the composition produces a band gap suitable for low intensity light harvesting, and wherein the blend has significant absorption of visible light between 380 nm and 940 nm suitable for low intensity light harvesting. Embodiment 47 is the composition of any one of embodiments 30 to 46, wherein the composition is provided in the form of a bulk material or a film.

Embodiment 48 is a composition comprising at least two electron acceptor materials A₁ and A₂, and at least an electron donor material D₁, wherein the electron acceptor materials A₁ and A₂, are of Formula Ia and/or Ib:

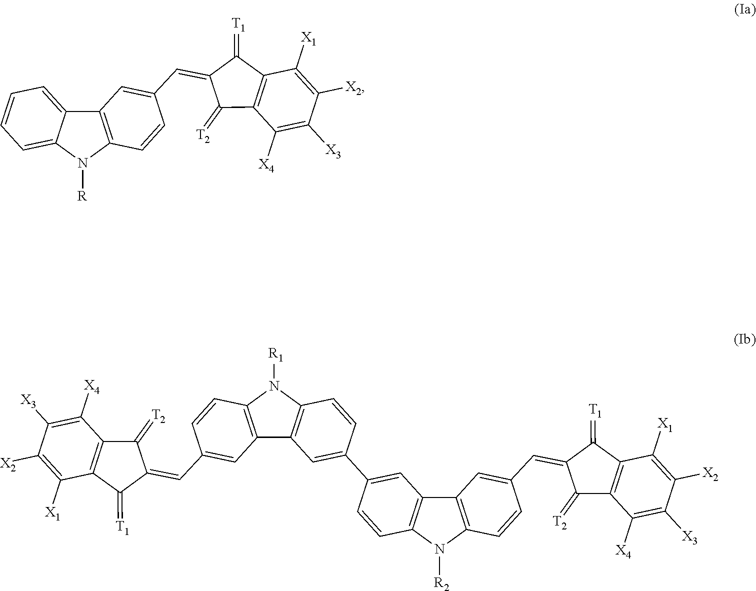

wherein R, R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN. Embodiment 49 is the composition of embodiment 48, wherein at least one of the two electron acceptor materials A₁ and A₂ has the structure of Formula Ila:

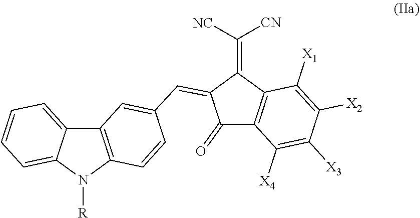

wherein R is a linear or branched C₆₋₁₅-alkyl; and X₁, X₂, X₃ and X₄ are each independently H, F, Cl or Br. Embodiment 50 is the composition of embodiment 48, wherein at least one of the two electron acceptor materials A₁ and A₂ has the structure of Formula IIIa:

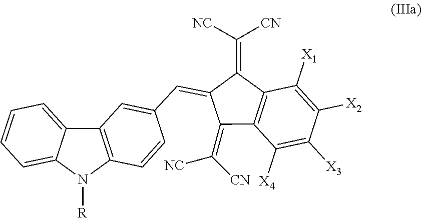

wherein R is a linear or branched C₆₋₁₅-alkyl; and X₁, X₂, X₃ and X₄ are each independently H, F, Cl or Br. Embodiment 51 is the composition of embodiment 48, wherein at least one of the two electron acceptor materials A₁ and A₂ has the structure of Formula IVa:

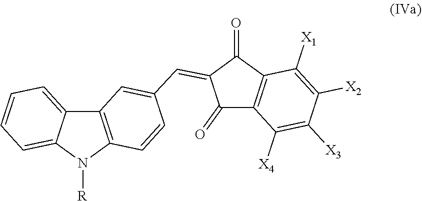

wherein R is a linear or branched C₆₋₁₅-alkyl; and X₁, X₂, X₃ and X₄ are each independently H, F, Cl or Br. Embodiment 52 is the composition of embodiment 48, wherein at least one of the two electron acceptor materials A₁ and A₂ has the structure of Formula IIb:

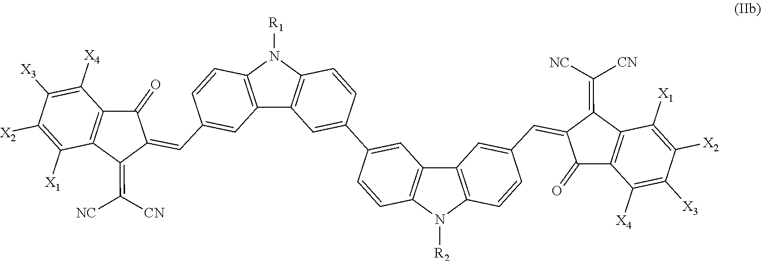

wherein R₁ and R₂ are each independently a linear or branched C₆₋₁₅-alkyl; and X₁, X₂, X₃ and X₄ are each independently H, F, Cl or Br. Embodiment 53 is the composition of embodiment 48, wherein at least one of the two electron acceptor materials A₁ and A₂ has the structure of Formula IIIb:

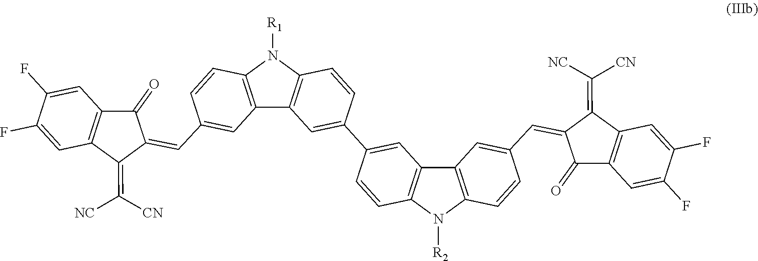

wherein R₁ and R₂ are each independently a linear or branched C₆₋₁₅-alkyl. Embodiment 54 is the composition of embodiment 48, wherein at least one of the two electron acceptor materials A₁ and A₂ has the structure of Formula IVb:

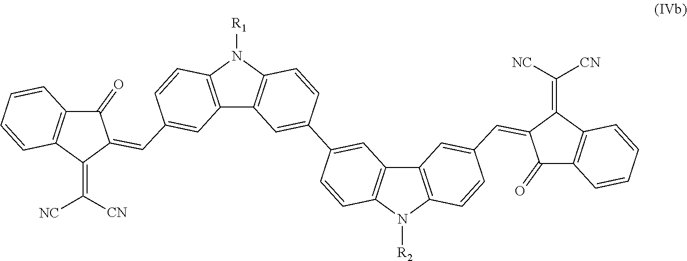

wherein R₁ and R₂ are each independently a linear or branched C₆₋₁₅-alkyl. Embodiment 55 is the composition of any one of embodiments 48 to 54, wherein the electron donor material D₁ comprises a p-type organic semiconductor material. Embodiment 56 is the composition of any one of embodiments 48 to 55, wherein the electron donor material D₁ comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT). Embodiment 57 is the composition of any one of embodiments 48 to 56, wherein the composition comprises a mass ratio (w/w) of acceptor material to donor material ranging from about 0.5:1.0 to about 1.0:0.5. Embodiment 58 is the composition of any one of embodiments 48 to 57, wherein the composition is slot die coated. Embodiment 59 is the composition of any one of embodiments 48 to 57, wherein the composition is spin coated. Embodiment 60 is the composition of any one of embodiments 48 to 59, wherein the composition produces a band gap suitable for low intensity light harvesting, and wherein the blend has significant absorption of visible light between 380 nm and 940 nm suitable for low intensity light harvesting. Embodiment 61 is the composition of any one of embodiments 48 to 60, wherein the composition is provided in the form of a bulk material or a film.

Embodiment 62 is the use of a compound as defined in any one of embodiments 1 to 11 as an electron acceptor compound.

Embodiment 63 is an organic solar cell comprising an anode and a cathode, and a photoactive layer between the anode and the cathode, wherein the photoactive layer comprises an electron donor/acceptor material, wherein the electron acceptor material is as defined in any one of embodiments 1 to 11. Embodiment 64 is the organic solar cell of embodiment 63, wherein the electron donor material comprises a p-type organic semiconductor material. Embodiment 65 is the organic solar cell of embodiment 63 or 64, wherein the electron donor material comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT). Embodiment 66 is the organic solar cell of any one of embodiments 63 to 65, wherein the photoactive layer comprises a mass ratio (w/w) of acceptor material to donor material ranging from about 0.5:1.0 to about 1.0:0.5. Embodiment 67 is the organic solar cell of any one of embodiments 63 to 66, wherein the photoactive layer is slot die coated. Embodiment 68 is the organic solar cell of any one of embodiments 63 to 66, wherein the photoactive layer is spin coated. Embodiment 69 is the organic solar cell of any one of embodiments 63 to 68, wherein one of the cathode and the anode comprises one of indium tin oxide (ITO), indium-doped zinc oxide (IZO), tin oxide (SnO), aluminum doped zinc oxide (AZO), or gallium-doped zinc oxide (GZO), and the other of the cathode and the anode includes one of aluminum (Al), silver (Ag), gold (Au), or lithium (Li). Embodiment 70 is the organic solar cell of any one of embodiments 63 to 69, wherein the photoactive layer produces a band gap suitable for low intensity light harvesting, and wherein the photoactive layer has significant absorption of visible light between 380 nm and 940 nm suitable for low intensity light harvesting. Embodiment 71 is the organic solar cell of any one of embodiments 63 to 70, wherein the photoactive layer is provided in the form of a bulk material or a film.

Embodiment 72 is an electronic device comprising a heterojunction, wherein the heterojunction comprises a blend comprising an electron donor and an electron acceptor material, wherein the electron acceptor material is as defined in any one of embodiments 1 to 11. Embodiment 73 is the electronic device of embodiment 72, wherein the electron donor material comprises a p-type organic semiconductor material. Embodiment 74 is the electronic device of embodiment 72 or 73, wherein the electron donor material comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT). Embodiment 75 is the electronic device of any one of embodiments 72 to 74, wherein the heterojunction comprises a mass ratio (w/w) of acceptor material to donor material ranging from about 0.5:1.0 to about 1.0:0.5. Embodiment 76 is the electronic device of any one of embodiments 72 to 75, wherein the blend is slot die coated. Embodiment 77 is the electronic device of any one of embodiments 72 to 75, wherein the blend is spin coated. Embodiment 78 is the electronic device of any one of embodiments 72 to 77, wherein the blend produces a band gap suitable for low intensity light harvesting, and wherein the blend has significant absorption of visible light between 380 nm and 940 nm suitable for low intensity light harvesting. Embodiment 79 is the electronic device of any one of embodiments 72 to 78, wherein the blend is provided in the form of a bulk material or a film. Embodiment 80 is the electronic device of embodiment 72, wherein the device is a photovoltaic cell, an organic transistor, a light emitting diode, or a photodetector.

Embodiment 81 is an organic semiconductor material, layer or component, comprising the composition as defined in any one of embodiments 12 to 29. Embodiment 82 is an organic semiconductor material, layer or component, comprising the composition as defined in any one of embodiments 30 to 47. Embodiment 83 is an organic semiconductor material, layer or component, comprising the composition as defined in any one of embodiments 48 to 61.

Embodiment 84 is the use of the composition as defined in any one of embodiments 12 to 29 as an organic semiconductor material, layer or component. Embodiment 85 is the use of the composition as defined in any one of embodiments 30 to 47 as an organic semiconductor material, layer or component. Embodiment 86 is the use of the composition as defined in any one of embodiments 48 to 61 as an organic semiconductor material, layer or component. Embodiment 87 is the use of the composition as defined in any one of embodiments 12 to 29 in an electronic device. Embodiment 88 is the use of the composition as defined in any one of embodiments 30 to 47 in an electronic device. Embodiment 89 is the use of the composition as defined in any one of embodiments 48 to 61 in an electronic device. Embodiment 90 is the use of any one of embodiments 84 to 86, wherein the electronic device is a photovoltaic cell, an organic transistor, a light emitting diode, or a photodetector.

Embodiment 91 is a bulk heterojunction (BHJ) formed from a composition as defined in any one of embodiments 12 to 29. Embodiment 92 is a bulk heterojunction (BHJ) formed from a composition as defined in any one of embodiments 30 to 47. Embodiment 93 is a bulk heterojunction (BHJ) formed from a composition as defined in any one of embodiments 48 to 61.

The foregoing and other advantages and features of the present disclosure will become more apparent upon reading of the following non-restrictive description of illustrative embodiments thereof, given by way of example only with reference to the accompanying drawings/figures.

### BRIEF DESCRIPTION OF THE DRAWINGS/FIGURES

In the appended drawings/figures:

FIG. 1 illustrates the chemical structures of various prior art carbazole-based non-fullerenes exhibiting a D-A general structure. Density functional theory (DFT) computations confirmed the D-A, not the A-D-A, nature for the non-fullerenes (D-A)2 and (D-A)3.

FIG. 2 illustrates the chemical structures of electron acceptor materials A₁ (MCz and MCzM) in accordance with embodiments of the present disclosure.

FIG. 3 illustrates the ORTEP representation of an electron acceptor material A₁ (MCz) in accordance with an embodiment of the present disclosure (A); and the crystal packing along the c-axis showing the spatial arrangement of each single stacked molecule; the crystal packing showing a staircase configuration where a head-to-tail orientation is adopted by the cofacially placed molecules (B).

FIG. 4 illustrates the cyclic voltammogram of an electron acceptor material A₁ (MCz) in CH₂Cl₂ (scan rate=50 mV/s) in accordance with an embodiment of the present disclosure (A); and the energy diagram illustrating the HOMO and LUMO energies of MCz (based on Eₒₓ^{onset} and E_{red}^{onset} values) and PTB7-Th (B).

FIG. 5 illustrates the absorption, emission and excitation spectra of an electron acceptor material A₁ (MCz) in accordance with an embodiment of the present disclosure: top left: in DCM at 298K; top right: in 2-MeTHF at 298K; bottom left: in solid state at 298K; and bottom right: in 2-MeTHF at 77K. The absorptivity of MCz at 520 nm is 46700 M⁻¹cm⁻¹.

FIG. 6 illustrates molecular orbital representations of the frontier orbitals of an electron acceptor material A₁ (MCz) (H=HOMO; L=LUMO) using the solvent field CH₂Cl₂ in accordance with an embodiment of the present disclosure.

FIG. 7 illustrates a simulated absorption spectrum of an electron acceptor material A₁ (MCz) using time dependent density functional theory (TDDFT) calculations, in accordance with an embodiment of the present disclosure. The bar graph under the absorption spectrum illustrates the calculated oscillator strength (f) and calculated positions of the first 100 electronic transitions for MCz. An arbitrary thickness of 1000 cm⁻¹ is assigned to each bar to generate the simulated spectrum.

FIG. 8 illustrates: (A) the emission decay of MCz in 2-MeTHF at 298K (emission decay; residuals; instrument response function (IRF); and best fit); Multi-exponential analysis yields τₚ=0.25 μs (16.2%), 1.67 μs (83.8%); χ²=1.08; Inset, Multi-exponential analysis; λ_{exc}=477 nm. (B) the emission decay of MCz in 2-MeTHF at 77K (emission decay; residuals; instrument response function (IRF); and best fit); Multi-exponential analysis yields τₚ=3.16 μs (100%); χ²=1.01; Inset, Multi-exponential analysis; λ_{exc}=477 nm. (C) the emission decay of MCz in solid state at 298K (emission decay; residuals; instrument response function (IRF); and best fit); Multi-exponential analysis yields τp=0.13 μs (28.77%), 0.46 μs (37.31%), 1.99 μs (33.92%); χ²=1.01; Inset, Multi-exponential analysis; λ_{exc}=477 nm.

FIG. 9 illustrates the chemical structures of electron acceptor materials A₁ (MDCzM-4F and MDCzM-4H) in accordance with embodiments of the present disclosure.

FIG. 10 illustrates absorption, and photoluminescence emission and excitation spectra for MDCzM-4F and P3HT (A); and absorption, and photoluminescence emission and excitation spectra for MDCzM-4H and P3HT (B), in accordance with an embodiment of the present disclosure.

FIG. 11 illustrates the photoluminescence quenching for P3HT, MDCzM-4F, and P3HT:MDCzM-4F (1:1) (A); P3HT, MDCzM-4H, and P3HT:MDCzM-4H (1:1) (B), in accordance with an embodiment of the present disclosure. No significant signal (i.e.; other than blank instrument response) was gathered for the P3HT:MDCzM-4F(1:1) and P3HT:MDCzM-4H(1:1) blends. There is significant photoluminescence quenching, most probably due to electron transfer in the P3HT:MDCzM-4F(1:1) and P3HT:MDCzM-4H(1:1) blends, rendering these donor/acceptor blends good candidates for use in organic solar cells.

FIG. 12 illustrates the absorption spectrum for the P3HT:MDCzM-4F (1:1) blend (A); and the P3HT:MDCzM-4H (1:1) blend (B), in accordance with embodiments of the present disclosure.

FIG. 13 illustrates the photoluminescence decay of MDCzM-4F in accordance with an embodiment of the present disclosure.

### DETAILED DESCRIPTION

### Glossary

In order to provide a clear and consistent understanding of the terms used in the present disclosure, a number of definitions are provided below. Moreover, unless defined otherwise, all technical and scientific terms as used herein have the same meaning as commonly understood by one of ordinary skill in the art to which this specification pertains.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±1% of the modified term if this deviation would not negate the meaning of the word it modifies.

The term “polymer” as used herein generally includes, but is not limited to, homopolymers and copolymers, such as for example block, random and alternating copolymers.

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound.

As used herein, fill factor (FF) is the ratio (given as a percentage) of the actual maximum obtainable power, (Pm or Vmp*Jmp), to the theoretical (not actually obtainable) power, (Jₛₑ*V_{oc}). Accordingly, the FF can be determined using the equation FF=(Vmp*Jmp)/(Jₛₑ*V_{oc}) where Jmp and Vmp represent the current density and voltage at the maximum power point (Pm), respectively, this point being obtained by varying the resistance in the circuit until J*V is at its greatest value; and Jₛₑ and V_{oc} represent the short circuit current and the open circuit voltage, respectively. The FF is a key parameter in evaluating the performance of solar cells.

As used herein, the open-circuit voltage (V_{oc}) is the difference in the electrical potentials between the anode and the cathode of a device when there is no external load connected.

As used herein, the power conversion efficiency (PCE) of a solar cell is the percentage of power converted from absorbed light to electrical energy. The PCE of a solar cell can be calculated by dividing the maximum power point (Pm) by the input light irradiance (E, in W/m²) under standard test conditions (STC) and the surface area of the solar cell (Ac in m²). STC typically refers to a temperature of 25° C. and an irradiance of 1000 W/m² with an air mass 1.5 (AM 1.5) spectrum.

As used herein, the term “active layer” will be understood to mean a blend of electron donor and electron acceptor materials that responds, to excitation.

As used herein, the term “absorption” will be understood to mean the ability or the process by which a material absorbs some or all of the incoming light energy.

As used herein, the term “excitation” will be understood to mean the process by which the energetic state of a material is changed using means such as light.

In an aspect, the present disclosure relates to binary compositions comprising an electron acceptor material A₁ and an electron donor material D₁, wherein the electron acceptor material A₁ is of Formula Ia:

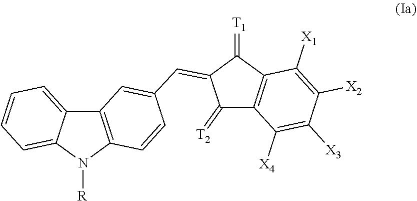

wherein R is a branched C₆₋₁₅-alkyl; X₁, X₂, X₃ and X₄ are each independently H, F, Cl or Br; and T₁ and T₂ are each independently ═O, ═C(CN)₂, or ═CHCN. In an embodiment of the present disclosure, the binary composition comprises a MCz:PTB7-Th system. In an embodiment of the present disclosure, the electron acceptor material A₁ has the structure:

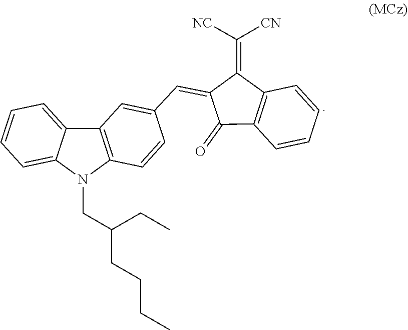 [SMILES: CCCCC(CC)Cn1c2ccccc2c2cc(/C=C3\C(=O)c4ccccc4C3=C(C#N)C#N)ccc21]

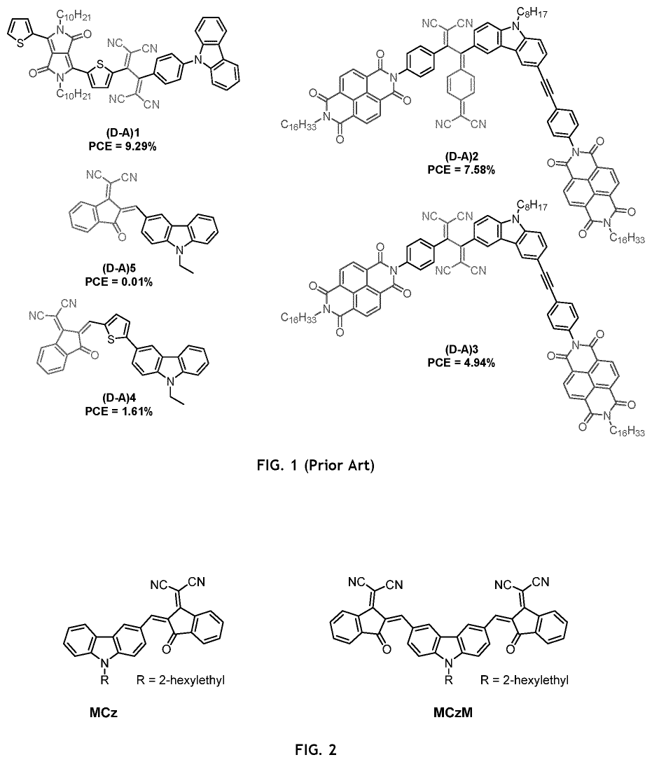

The use of MCz advantageously provided for improved PCEs for outdoor (AM=1.5) and indoor (800 Lux, 3000K) applications, relative to those reported for the compounds illustrated in FIG. 1. Notably, a PCE of 9.43% was observed for MCz. Surprisingly, changing the ethyl group (D-A)5 for a 2-ethylhexyl group (MCz) dramatically improved the PCE from 0.01% to 9.43%. MCzM was previously investigated and was also shown to exhibit good OSC performances.^{[21]} Simplifying the MCzM structure to MCz (FIG. 2) surprisingly did not adversely impact the PCE or OSC performance.

The synthesis of MCz, in accordance with an embodiment of the present disclosure, is illustrated in Scheme 1.

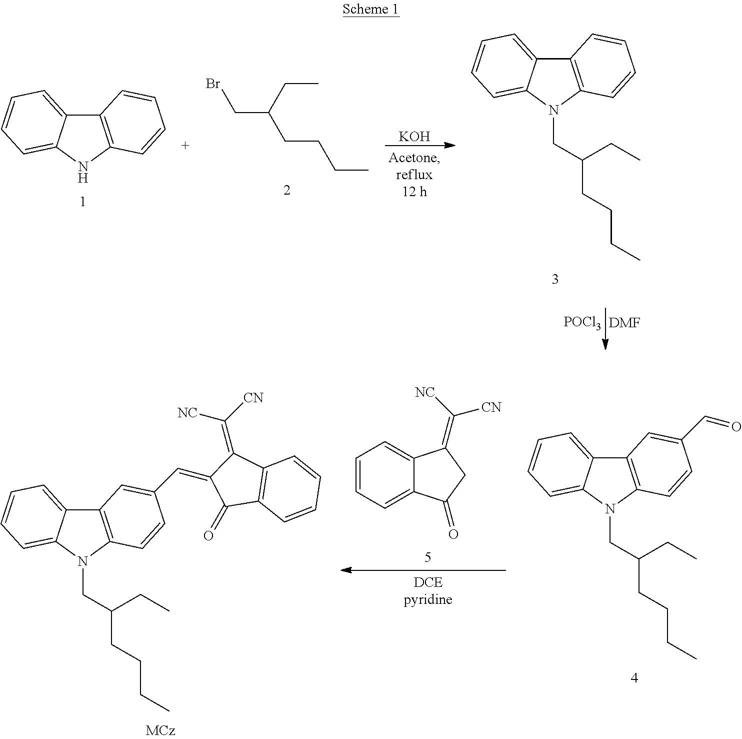

2-(3-oxo-2,3-dihydro-1H-inden-1-ylidene)malononitrile, 5, was prepared according to a known literature procedure.^{[21]} Commercially available 1H-indene-1,3(2H)-dione was reacted with malononitrile in a Knoevenagel condensation to afford the 2-(3-oxo-2,3-dihydro-1H-inden-1-ylidene)malononitrile moiety, 5. Concurrently, to increase the solubility of all subsequent compounds, carbazole (1) was alkylated with 2-ethylhexyl bromide (2) in quantitative yield to afford the 9-(2-ethylhexyl)-9H-carbazole moiety, 3. The mono- and bis formylation of the 9-(2-ethylhexyl)-9H-carbazole in position-3 and/or -6, was subsequently performed. The mono-formylated carbazole (4) was isolated or prepared separately by varying the ratio of POCl₃ in DMF, with isolated yields of −72%. The mono-formylated carbazole (4) was reacted with 2 equivalents of malononitrile 5 to afford MCz in 37% isolated yield. The bis-formylated carbazole was similarly reacted to prepare MCzM.

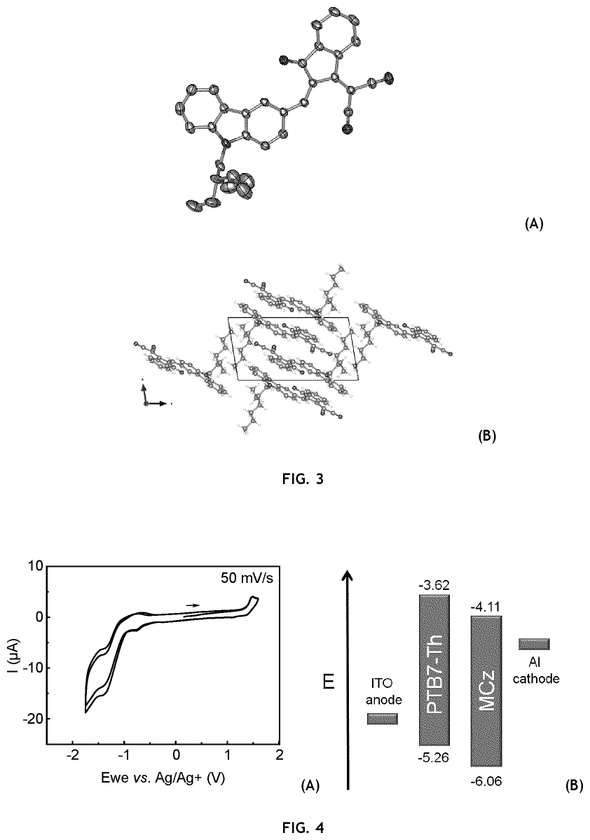

Single crystals suitable for X-ray diffraction were obtained from a hot mixture of acetonitrile/toluene (1:1) followed by slow evaporation over a period of three days (FIG. 3). The X-ray structure reveals a quasi-planar conformation in which the dihedral angle made by the malononitrile and carbazole average plane is 3.8°. This conformation is rendered possible because the N≡C—C—C≡N moieties are positioned away from the carbazole units. The 2-ethylhexyl chain is placed quasi-perpendicular to the carbazole plane forming an angle of 89.22° between the average chain axis and carbazole plane. The crystal packing shows a staircase configuration where a head-to-tail orientation is adopted by the cofacially placed molecules. The interplanar separation is 3.380 Å, suggesting good 71-71 contact between the respective MCz molecules in the solid state.

The cyclic voltammogram (CV) of MCz was recorded to localize the HOMO and LUMO (FIG. 4A). The electrochemical data are illustrated in Table 1. In comparison with the electron donor PTB7-Th, the HOMO and LUMO of MCz are at lower energy levels than those of the conjugated polymer, which makes these materials adequate for downhill electron transfer in bulk heterojunction organic solar cells (FIG. 4B). In an embodiment of the present disclosure, the materials were chosen in part to cover a broad region of the indoor LED emission spectrum.

TABLE 1
Electrochemical data, HOMO and LUMO
energies, and band gaps for MCz.

| | MCz |
|---|---|
| Eₒₓ (V)^{a} | +1.46 |
| Eₒₓ^{onset} (V)^{a} | +1.35 |
| E_{HOMO} (eV)^{b} | −6.06 |
| E_{red} (V)^{a} | −0.74 |
| E_{red}^{onset} (V)^{a} | −0.60 |
| E_{LUMO} (eV)^{b} | −4.11 |
| E_{g} (eV)^{c} | 2.20 |
| E_{g}^{onset} (eV)^{c} | 1.95 |
| E_{g}^{av} (eV)^{c} | 2.08 |
| E_{g}^{opt} (eV)^{c} | 2.15 |

^{a}Eₒₓ = oxidation peak potential;
Eₒₓ^{onset} = oxidation onset;
E_{red} = reduction peak potential;
E_{red}^{onset} = reduction onset;
^{b}E_{HOMO} = −(Eₒₓ + 4.71);
E_{LUMO} = −(E_{red} + 4.71);
^{c}E_{g} = (Eₒₓ − E_{red});
E_{g}^{onset} = (Eₒₓ^{onset} − E_{red}^{onset});
E_{g}^{av} = (E_{g} + E_{g}^{onset})/2;
E_{g}^{opt} = optical E_{g}

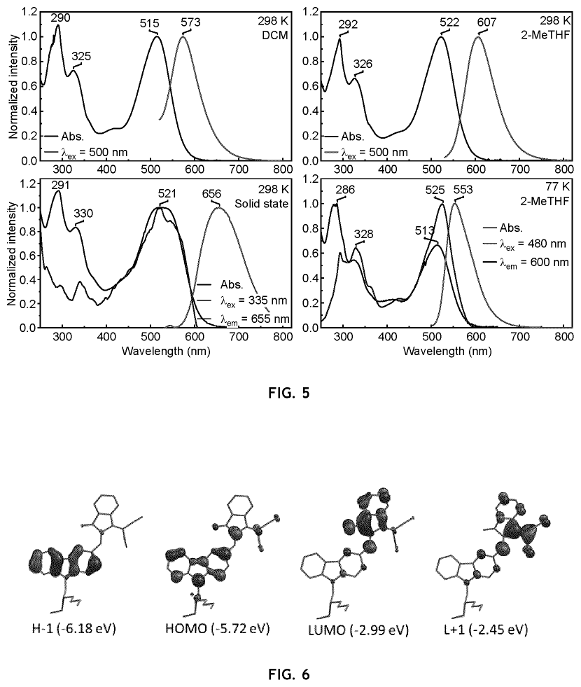

The absorption spectra of MCz exhibit two main regions at 250-400 and 400-600 nm respectively (FIG. 5). The frontier molecular orbitals (HOMO and LUMO orbitals) of MCz are illustrated in FIG. 6. The LUMO and LUMO+1 exhibit atomic contributions mainly located on the π-system of the 2-(3-oxo-2,3-dihydro-1H-inden-1-ylidene)malononitrile moiety (i.e., the electron withdrawing moiety), whereas those for the HOMO and HOMO-1 are mostly located on the π-system of the carbazole moiety (i.e., the electron donating moiety) (Table 2). Electronic transitions between these HOMO and LUMO molecular orbitals lead to charge transfer excited states, namely N-alkylcarbazole→malononitrile moiety, demonstrating the push-pull character of MCz.

TABLE 2
Relative atomic contributions (%) for various
fragments of the frontier MOs of MCz.^{a}

| Fragment | HOMO − 1 | HOMO | LUMO | LUMO + 1 |
|---|---|---|---|---|
| carbazole core | 94.24 | 72.02 | 15.36 | 10.63 |
| malononitrile moiety | 5.22 | 22.19 | 84.05 | 89.01 |
| alkyl chain | 0.54 | 5.79 | 0.59 | 0.36 |

^{a}The values in bold represent the largest contributions.

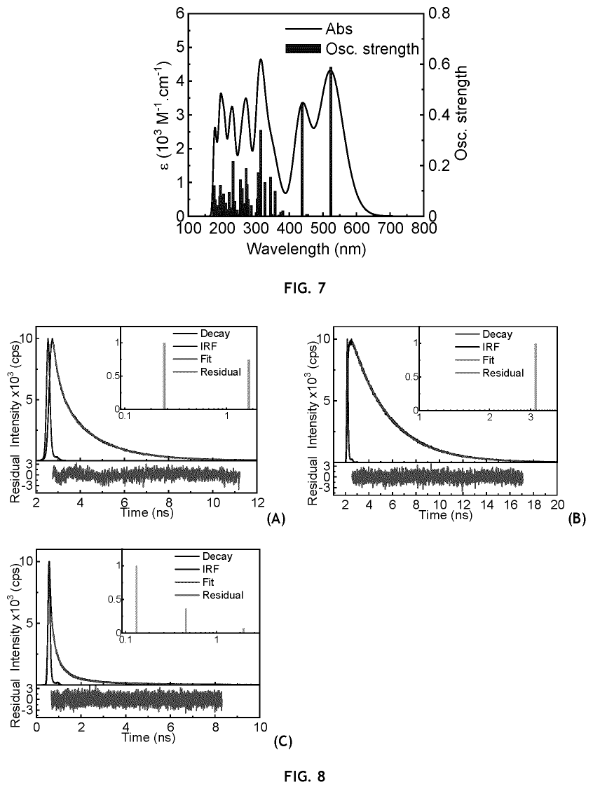

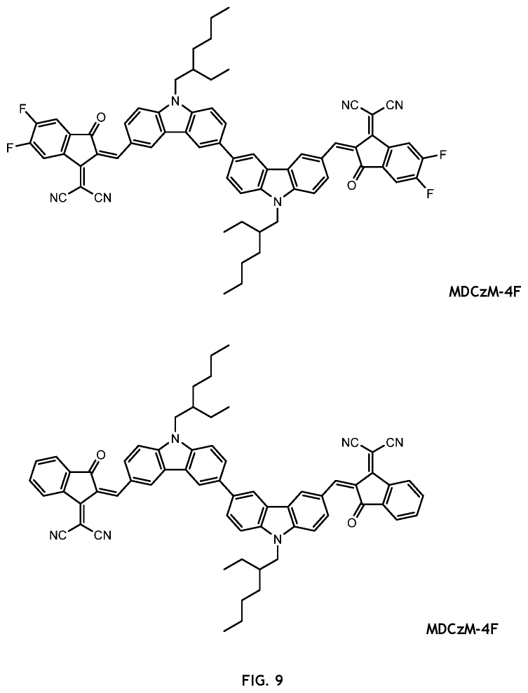

TDDFT computations place the lowest energy spin-allowed transition at 523 nm (Table 3). This value compares favorably to the experimental band maximum at 520 nm (FIG. 5, top). This transition is composed of 98% of a HOMO→LUMO transition and the calculated oscillator strength (f) is large, which is consistent with the observed large absorptivity (s=46700 M⁻¹ cm⁻¹). This transition is well-isolated from the next two transitions at 455 and 438 nm respectively, which are composed of HOMO→LUMO+1 and HOMO-1→LUMO transitions, in different proportions. These latter two electronic transitions also lead to charge excited states, namely N-alkylcarbazole→malononitrile moiety. Finally, upon computing the 100 first electronic transitions, a simulated spectrum was generated (FIG. 7). The simulated spectrum resembled that of the experimental spectrum (FIG. 5), confirming that the calculation method is adequate to describe the push-pull ability of MCz, and thus corroborates the charge transfer (CT) assignments.

TABLE 3
Calculated position, oscillator strength (f) and major contributions
(%) of the three first spin-allowed electronic transitions for MCz.

| No. | λ (nm) | f | Major contributions (%) |
|---|---|---|---|
| 1 | 523.4 | 0.5897 | HOMO→LUMO (98%) |
| 2 | 455.1 | 0.0079 | H − 1→LUMO (83%), HOMO→L + 1 (15%) |
| 3 | 438.2 | 0.4484 | H − 1→LUMO (15%), HOMO→L + 1 (81%) |

MCz is strongly emissive both in solution and in the solid state (FIG. 5). The fluorescence lifetime (TF) data for MCz are illustrated in Table 4. The nanosecond (ns) time scale is consistent with the emission arising from the singlet state (i.e., fluorescence). Multiple exponential decays are observed for samples at 298K (FIG. 8). Speculatively, rotation around the C—C single bond between the carbazole moiety (i.e., the electron donating moiety (D)) and the malononitrile moiety (i.e., the electron withdrawing moiety (A)) could generate multiple conformers. At 77K, the lowering of the thermal energy could favor the lowest energy conformation. In CH₂Cl₂, at 298K, the TF is <0.10 ns (i.e., at the limit of the measurement; the full width at half maximum (FWHM) of the excitation pulse being ˜100 μs). A very weak fluorescence signal could be observed in CH₂Cl₂ at 298K, which can be readily explained by the quenching effect of this solvent. The lower τ_{F} (<0.10 ns) is accompanied by lower fluorescence intensity. Chlorinated solvents such as CHCl₃ and CH₂Cl₂ are known to often quench fluorescence. Both outcomes indicate quenching, which is consistent with the known proficiency of carbazoles to photooxidize in the presence of halocarbons.

TABLE 4
Fluorescence lifetime of MCz under various
conditions (λₑₓ = 477 nm).

| Conditions | τ_{F} (ns) | f (%) | χ² |
|---|---|---|---|
| 2-MeTHF (298K) | 0.25; 1.67 | 16.2; 83.8 | 1.082 |
| 2-MeTHF (77K) | 3.16 | 100 | 1.011 |
| solid state (298K) | 0.13; 0.46; 1.99 | 28.8; 37.3; 33.9 | 1.014 |

fᵢ(%) = (Bᵢτᵢ)/Σ(Bᵢτᵢ) · 100%;
Iₑ(t) = B₁exp(−t/τ₁) + B₂exp(−t/τ₂) + B₃exp(−t/τ₃) + . . .

Devices comprising a binary composition comprising an electron acceptor material A₁ (MCz or MCzM) and an electron donor material D₁ (PTB7-Th; HOMO=−5.26 eV and LUMO=3.62 eV) were prepared. The optical spectra extended up to 780 nm. The photovoltaic metrics are illustrated in Tables 5 and 6.

TABLE 5
Photovoltaic metrics - Indoor (AM = 1.5; G = 100 mW/cm²).

| Active layer | V_{oc} (V) | J_{sc} (mA/cm²) | FF | PCE (%) |
|---|---|---|---|---|
| MCzM: PTB7-Th | 1.02 | 17.67 | 0.66 | 11.72 |
| MCz: PTB7-Th | 0.82 | 18.26 | 0.63 | 9.43 |

Active layers based on the binary composition (MCz:PTB7-Th) achieved a maximum PCE of 9.43% with the following photovoltaic parameters: J_{sc}=18.26 mA/cm², V_{oc}=0.82 V, FF=0.63. These results compare well with the MCzM:PTB7-Th binary composition. Interestingly, transitioning from a MCzM:PTB7-Th binary composition to a MCz:PTB7-Th binary composition showed little loss in device performance (PDE of 11.72% vs. 9.43%).

TABLE 6
Photovoltaic metrics - Indoor LED (800 Lux; 3000K).

| Active layer | V_{oc} (V) | J_{sc} (μA/cm²) | FF | PCE (%) |
|---|---|---|---|---|
| MCzM: PTB7-Th | 0.88 | 81.08 | 0.65 | 18.55 |
| MCz: PTB7-Th | 0.74 | 91.85 | 0.62 | 16.86 |

Active layers based on the binary composition (MCz:PTB7-Th) achieved a maximum PCE of 16.86% with the following photovoltaic parameters: J_{sc}=91.85 mA/cm², V_{oc}=0.74 V, FF=0.62. These results compare well with the MCzM:PTB7-Th binary composition. Interestingly, transitioning from a MCzM:PTB7-Th binary composition to a MCz:PTB7-Th binary composition again showed little loss in device performance (PDE of 16.86% vs. 18.55%).

The synthesis of MDCzM-4F and MDCzM-4H, in accordance with an embodiment of the present disclosure, is illustrated in Scheme 2.

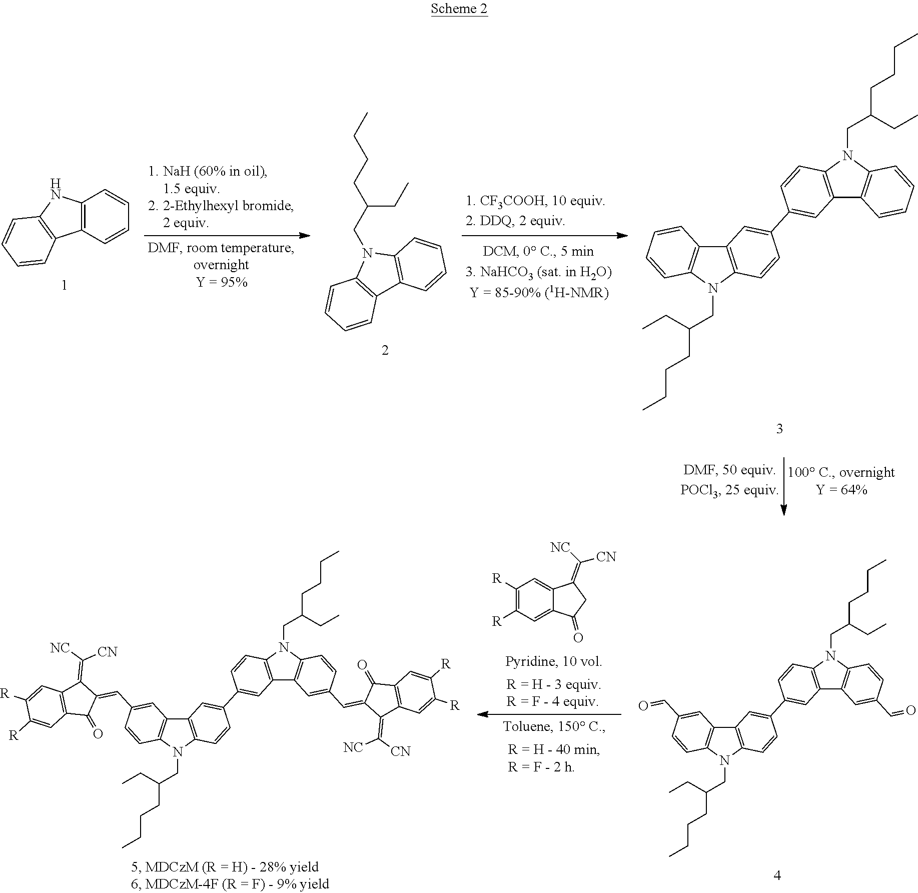

In an aspect, the present disclosure relates to binary compositions comprising an electron acceptor material A₁ and an electron donor material D₁, wherein the electron acceptor material A₁ is of Formula Ib:

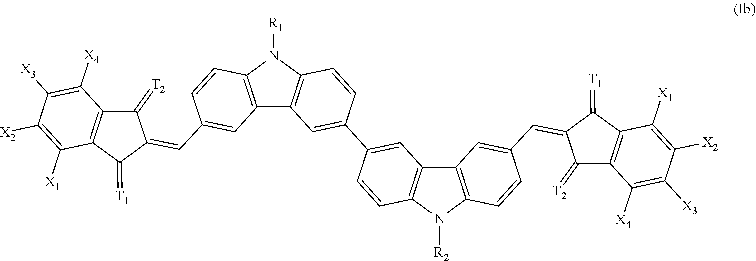

wherein R₁ and R₂ are each independently a linear or branched C₆₋₁₅-alkyl; X₁, X₂, X₃ and X₄ are each independently H, F, Cl or Br; and T₁ and T₂ are each independently ═O, ═C(CN)₂, or ═CHCN.

In an embodiment of the present disclosure, the binary composition comprises a MDCzM-4F:P3HT system. In an embodiment of the present disclosure, the binary composition comprises a MDCzM-4H:P3HT system. In an embodiment of the present disclosure, the electron acceptor material A₁ has either of the following structures:

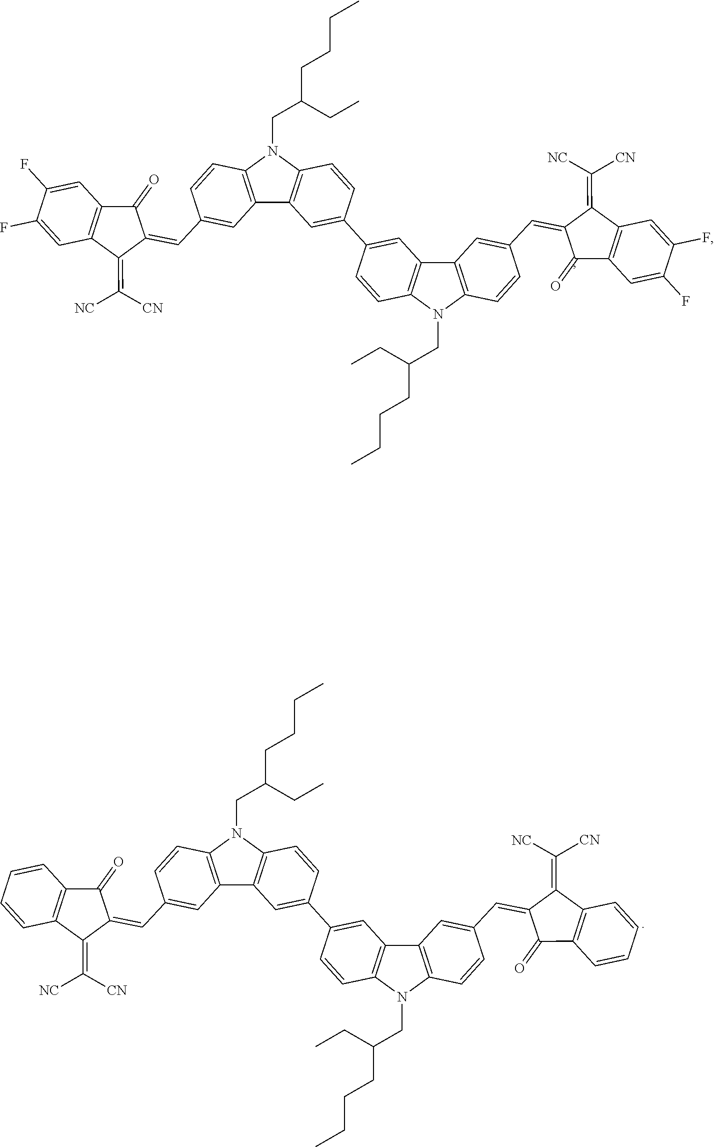 [SMILES: CCCCC(CC)Cn1c2ccc(/C=C3\C(=O)c4cc(F)c(F)cc4C3=C(C#N)C#N)cc2c2cc(-c3ccc4c(c3)c3cc(/C=C5\C(=O)c6cc(F)c(F)cc6C5=C(C#N)C#N)ccc3n4CC(CC)CCCC)ccc21 ; CCCCC(CC)Cn1c2ccc(/C=C3\C(=O)c4ccccc4C3=C(C#N)C#N)cc2c2cc(-c3ccc4c(c3)c3cc(/C=C5\C(=O)c6ccccc6C5=C(C#N)C#N)ccc3n4CC(CC)CCCC)ccc21]

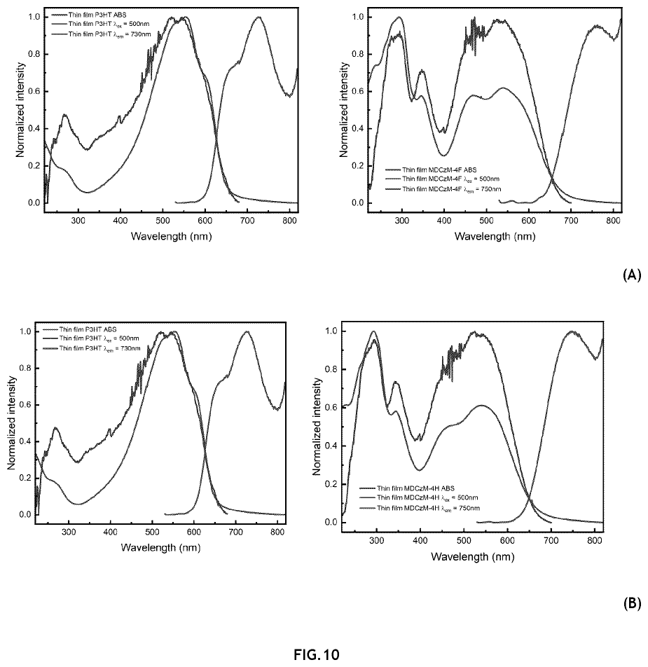

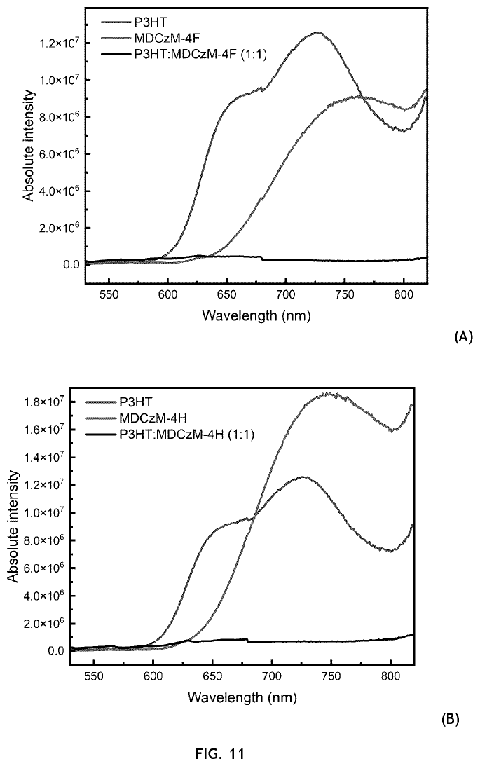

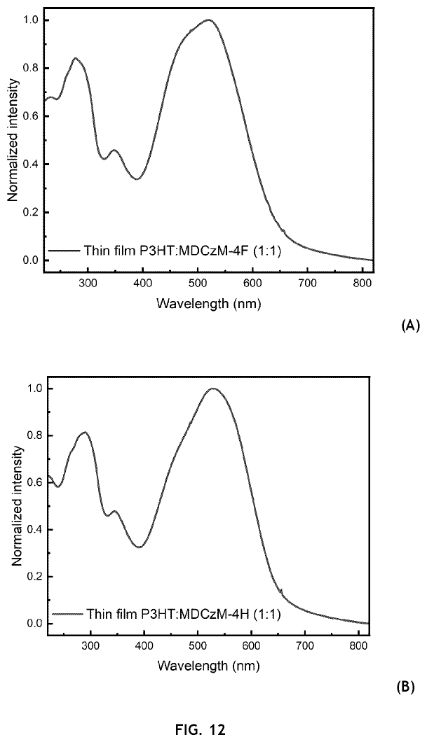

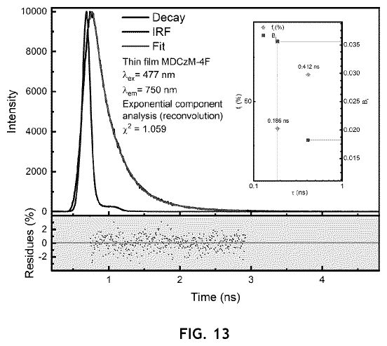

Photophysical data were acquired for both P3HT and MDCzM-4F and are illustrated in Table 7 as well as FIGS. 10A and 11A.

TABLE 7
Photophysical data for P3HT and MDCzM-4F

| | λ_{abs} (nm) | λₑₘ (nm) | τ_{TCSPC} (ps) | Φ_{PL} |
|---|---|---|---|---|
| P3HT | 554 | 666; 729 | N/A | N/A |
| MDCzM-4F | 295; 346; 467; 538 | 760 | 306 | N/A |

N/A: Not applicable (measurement under the limit of detection)

Photophysical data were acquired for both P3HT and MDCzM-4H and are illustrated in Table 8 as well as FIGS. 10B and 11B.

TABLE 8
Photophysical data for P3HT and MDCzM-4H

| | λ_{abs} (nm) | λₑₘ (nm) | τ_{TCSPC} (ps) | Φ_{PL} |
|---|---|---|---|---|
| P3HT | 554 | 666; 729 | N/A | N/A |
| MDCzM-4H | 294; 345; 469; 541 | 747 | N/A | N/A |

N/A: Not applicable (measurement under the limit of detection)

### EXPERIMENTAL

A number of non-limiting examples are provided in the following sections, illustrating the preparation of novel carbazole-based non-fullerenes, binary and ternary compositions, and devices comprising same, in accordance with various embodiments of the present disclosure. The following non-limiting examples are illustrative of the present disclosure.

### Materials

PTB7-Th (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) was purchased from Odessa and was used as received. MCzM was synthesized according to a literature procedure.^{[21]}

### Preparation of MCz

2-(3-oxo-2,3-dihydro-1H-inden-1-ylidene)malononitrile (575 mg, 2.96 mmol) and 9-(2-ethylhexyl)-9H-carbazole-3-carbaldehyde (492 mg, 1.6 mmol) were dissolved under stirring in dichloroethane (50 mL) and pyridine (1.3 mL). The mixture was subsequently refluxed for one hour. After cooling down to room temperature, the crude material was purified by column chromatography on silica gel with EtOAc/hexane (1:3 to 1:4) as mobile phase to yield 290 mg (37.5%) of a dark red solid. ¹H NMR (400 MHz, CDCl₃) δ 9.21 (s, 1Hₐᵣₒ), 8.77 (s, 1Hₐᵣₒ), 8.67-8.65 (d, 1Hₐᵣₒ), 8.47-8.45 (d, 1Hₐᵣₒ), 8.21-8.19 (d, 1Hₐᵣₒ), 7.94-7.92 (m, 1Hₐᵣₒ), 7.76-7.72 (m, 2Hₐᵣₒ), 7.54-7.50 (t, 1Hₐᵣₒ), 7.42-7.40 (d, 2Hₐᵣₒ), 7.36-7.32 (t, 1Hₐᵣₒ), 4.17-4.15 (m, CH₂), 2.08-2.02 (quint, CH), 1.44-1.22 (m, 4CH₂), 0.94-0.91 (t, CH₃), 0.88-0.85 (t, CH₃). ¹³C NMR (101 MHz, CDCl₃) δ 187.14, 163.09, 149.64, 144.76, 141.71, 139.72, 137.50, 135.07, 134.56, 134.06, 129.55, 127.04, 125.65, 125.15, 124.51, 124.03, 123.76, 123.29, 121.18, 121.10, 114.95, 114.74, 110.01, 109.39, 69.78, 47.89, 39.57, 31.10, 28.91, 24.52, 23.14, 14.15, 11.01. A crystal of MCz for use in X-ray diffraction was obtained by dissolving the powder in a hot mixture of acetonitrile/toluene (1:1). Slow evaporation over 3 days afforded adequately sized purple needles suitable for X-ray diffraction.

### Preparation of MDCzM-4F and MDCzM-4F (Scheme 2)

### 9,9′-bis(2-ethylhexyl)-9H,9′H-3,3′-bicarbazole (3)

Compound 3 was prepared using trifluoroacetic acid (10 equiv.). Crude material (85-90% desired product 3-obtained as a mixture) was used directly in the next step.

### 9,9′-bis(2-Ethylhexyl)-9H,9′H-[3,3′-bicarbazole]-6,6′-dicarbaldehyde (4)

POCl₃ (8.4 ml, 25 equiv.) was carefully added to a mixture of 3 (2 g, 3.6 mmol) in DMF (7 ml, 25 equiv.) under Ar at room temperature while stirring. The reaction mixture was stirred at 100° C. for 2 h in a sealed Schlenk tube. TLC revealed plenty of starting material and mono aldehyde. Additional DMF (7 ml, 25 equiv.) was added, and the reaction continued overnight. The resulting reaction cake was dissolved in 250 ml of DCM after cooling to room temperature and carefully mixed with solid Na₂CO₃ and stirred for 5 h. The product was extracted with DCM (2×250 ml), the organic layers combined, washed with water and brine, dried over Na₂SO₄, filtered and concentrated. The crude product was adsorbed on silica, and subjected to chromatography (SiO₂ column) using DCM as the eluent to obtain 1.4 g of a pure product (Y=64%). ¹H-NMR (400 MHz, CDCl₃): δ 10.15 (s, 2H), 8.72 (d, J=1.29 Hz, 2H), 8.50 (d, J=1.54 Hz, 2H), 8.06 (dd, J₁=8.56 Hz, J₂=1.50 Hz, 2H), 7.92 (dd, J₁=8.49 Hz, J₂=1.74 Hz, 2H), 7.57 (d, J=8.58 Hz, 2H), 7.51 (d, J=8.57, 2H), 4.18-4.27 (m, 4H), 2.15 (hep, J=5.86 Hz, 2H), 1.51-1.28 (m, 16H), 0.99 (t, J=7.41 Hz, 6H), 0.91 (t, J=7.12 Hz, 6H). ¹³C-NMR (400 MHz, CDCl₃): δ 191.73, 144.98, 140.84, 134.21, 128.59, 127.15, 126.39, 124.18, 123.66, 123.19, 119.21, 110.09, 109.45, 47.88, 39.49, 31.01, 28.79, 24.42, 23.06, 14.06, 10.94.

### 2,2′-((2Z,2′Z)-((9,9′-bis(2-ethylhexyl)-9H,9′H-[3,3′-bicarbazole]-6,6′-diyl)bis(methanylylidene))bis(3-oxo-2,3-dihydro-1H-indene-2,1-diylidene))-imalononitrile, MDCzM (5)

A mixture of compound 4 (300 mg, 0.49 mmol), 2-(3-oxo-2,3-dihydro-1H-inden-1-ylidene)-malononitrile (285 mg, 3 equiv.) and pyridine (3 ml) in toluene (45 ml) was stirred in a sealed Schlenk tube at 150° C. for 40 min under Ar. The reaction mixture was then cooled to room temperature and concentrated. The crude material was triturated in EtOH and filtered. The resulting material was suspended in a large quantity of CHCl₃ and filtered. The filtrate was evaporated and purified by preparative TLC in 3 portions, using DCM/Hex=9/1 as an eluent to obtain 130 mg of a dark-violet product. R_{f}=0.94 (DCM) (Y=28%). ¹H-NMR (400 MHz, CDCl₃): δ 9.38 (s, 2H), 8.74 (s, 2H), 8.49 (d, J=7.70 Hz, 2H), 8.41 (dd, J₁=8.86 Hz, J₂=1.71 Hz, 2H), 8.38 (d, J=1.76 Hz, 2H), 8.01 (dd, J₁=7.15 Hz, J₂=1.48 Hz, 2H), 7.87 (dd, J₁=8.48 Hz, J₂=1.81 Hz, 2H), 7.65 (ddd, J₁=7.41 Hz, J₂=7.33 Hz, J₃=1.09 Hz, 2H), 7.60 (ddd, J₁=7.51 Hz, J₂=7.38 Hz, J₃=1.45 Hz, 2H), 7.49 (d, J=8.51 Hz, 2H), 7.44 (d, J=8.81 Hz, 2H), 4.28-4.16 (m, 4H), 2.17-2.06 (m, 2H), 1.50-1.30 (m, 16H), 0.99 (t, J=7.38 Hz, 6H), 0.92 (t, J=7.07 Hz, 6H). ¹³C-NMR (100 MHz, CDCl₃): δ 187.12, 162.59, 149.05, 144.86, 140.74, 139.42, 137.33, 134.74, 134.41, 134.36, 134.30, 129.44, 126.47, 125.47, 124.75, 124.60, 124.01, 123.83, 123.81, 119.54, 114.88, 114.67, 110.18, 109.42, 69.36, 47.90, 39.54, 31.03, 28.83, 24.45, 23.05, 14.07, 10.91.

### 2,2′-((2Z,2′Z)-((9,9′-bis(2-ethylhexyl)-9H,9′H-[3,3′-bicarbazole]-6,6′-diyl)bis(methanylylidene))bis(5,6-difluoro-3-oxo-2,3-dihydro-1H-indene-2,1-diylidene))dimalononitrile, MDCzM-4F (6)

A mixture of compound 4 (100 mg, 0.17 mmol), 2-(5,6-difluoro-3-oxo-2,3-dihydro-1H-inden-1-ylidene)-malononitrile (150 mg, 4 equiv.) and pyridine (1 ml) in toluene (8 ml) was stirred in a sealed Schlenk tube at 150° C. for 2 h under Ar. The reaction mixture was then cooled to room temperature and concentrated. The crude material was triturated in EtOH and filtered. The resulting black material was suspended in a large quantity of CHCl₃ and filtered. The filtrate was adsorbed on SiO₂, and subjected to chromatography (SiO₂ column) using DCM as the eluent. The product (51 mg) was then recrystallized from a CHCl₃/Hex mixture to afford 31 mg of a dark-violet solid. R_{f}=0.84 (DCM); Y=9%. ¹H-NMR (400 MHz, CDCl₃): δ 9.63 (s, 2H), 8.64 (s, 2H), 8.29 (s, 2H), 8.17 (d, J=8.67 Hz, 2H), 8.11-8.04 (m, 2H), 7.88 (dd, J₁=J₂=7.64 Hz, 2H), 7.82 (d, J=8.46 Hz), 7.51 (d, J=8.42 Hz, 4H), 4.35-4.26 (m, 4H), 2.20-2.12 (m, 2H), 1.50-1.32 (m, 16H), 1.02 (t, J=7.35 Hz, 6H), 0.94 (t, J=7.04, 6H). ¹⁹F-NMR (400 MHz, CDCl₃): δ −123.24, −123.50. ¹³C-NMR (100 MHz, CDCl₃): δ 184.91, 159.59, 155.39 (m), 152.94 (m), 148.52, 145.04, 140.91, 135.76 (dd, J₁=7.61 Hz, J₂=4.04 Hz), 135.33, 134.93 (dd, J₁=4 Hz, J₂=3 Hz), 134.51, 128.67, 126.47, 124.52, 124.36, 124.10, 123.77, 119.10, 114.42, 114.05, 113.84 (d, J=21.06 Hz), 112.88 (d, J=18.50), 110.36, 109.87, 48.13, 39.64, 31.09, 29.66, 28.79, 24.50, 22.94, 13.87, 10.77.

Thin films of P3HT and MDCzM-4H were prepared on thin quartz plates by depositing 50 μl of a 10 mg/mL solution while rotating at a speed of 3000 rpm for 30 seconds. A film of P3HT:MDCzM-4H(1:1) was obtained by subjecting a quartz plate to the same conditions using a 1:1 solution of the same total concentration. All thin films were used as is without further treatment.

Thin films of P3HT and MDCzM-4F were prepared on thin quartz plates by depositing 50 μl of a 10 mg/mL solution while rotating at a speed of 3000 rpm for 30 seconds. A film of P3HT:MDCzM-4F(1:1) was obtained by subjecting a quartz plate to the same conditions using a 1:1 solution of the same total concentration. All thin films were used as is without further treatment.

### Instruments

Solid state UV-vis spectra were recorded on a Varian Cary 50 spectrophotometer at 298 K and 77 K using a raised-angle transmittance apparatus and a homemade 77K sample-holder. Steady state emission and excitation spectra were measured on an Edinburgh Instruments FLS980 Phosphorimeter equipped with single monochromators. The steady state emission spectra were recorded using capillaries for the solid state, an NMR tube for the 77 K measurements, and an airtight 1 cm cuvette for measurements in solution at 298 K, which were prepared in a glove box. These spectra were corrected for instrument response. The phosphorescence lifetime measurements were performed with an Edinburgh Instruments FLS980 Phosphorimeter equipped with a “flash” pulsed lamp. The frequency of the pulse was adjusted from 1 to 100 Hz. All lifetime values were obtained from deconvolution and distribution lifetime analysis and multi-exponential analysis for comparison purposes. Photoluminescence quantum yields were obtained on a HORIBA FluoroF spectrophotometer coupled to an integration sphere. Slits were kept constant as_to obtain a Rayleigh diffusion intensity of 1,000,000 Cts at 500 nm.

### Electrochemistry

The cyclic voltammetry measurements for MCz were performed using an Ag/Ag⁺ (sat. KCl) reference electrode, a Pt mesh as counter electrode, and a Pt disc electrode as working electrode. The ferrocene/ferrocenium couple was used as an internal reference. The CV measurements were performed in distilled dichloromethane. All measurements were performed with an argon degassed 10-1 M solution of tetra-butylammonium hexafluorophosphate. All CVS measurements were made from a 10⁵ M solution in dichloromethane.

### Single Crystal X-Ray Diffraction

A purple needle-like specimen of C₃₃H₂₉N₃₀ (MCz) was mounted on a Bruker APEX-II DUO equipped with a nitrogen jet stream low-temperature system (Oxford Cryosystems). The X-ray source was using graphite monochromated Mo-Kα radiation (λ=0.71073 Å) from a sealed tube or Cu (λ=1.54186 Å) microfocus tube IμS with MX optics by Incoatec. The lattice parameters were obtained by the method of least-squares to determine the best fit to the optimized setting angles of the entire set of collected reflections. Intensity data were recorded as ϕ and ω scans. Data were reduced using SAINT v8.37A (Bruker, 2015) software and absorption corrections were carried out by SADABS-2016/2 (Bruker, 2016). The structure was solved using the Bruker SHELXTL Software Package. Refinement was performed using the shelxl-crystal structure refinement-multi-cpu version, George M. Sheldrick 1993-2018 version 2018/3 on the complete set of reflections. All non-hydrogen atoms were refined with anisotropic thermal parameters.

### Density Functional Theory Computations

The density functional theory (DFT) and time dependent density functional theory (TDDFT) calculations were performed with Gaussian 16 at the Universite de Sherbrooke with the Mammoth supercomputer supported by Calcul Québec. Crystallographic Information Files (CIF) from the X-ray crystal structure of MCz were used as the starting point for the calculations. The DFT (ground states) as well as TDDFT calculations were carried out using the B3LYP method. The 6-31g(d,p) basis set was used for all atoms. All calculations were carried out using a dichloromethane CPCM solvent field. No imaginary frequencies were observed validating the correct energy minimization after the optimization. The calculated absorption spectra were obtained using GaussSum 3.0. An isosurface was generated with a contour value of 0.0432; red lobes are positive and blue lobes are negative.

While the present disclosure has been described with reference to specific examples, it is to be understood that the disclosure is not limited to the disclosed examples. To the contrary, the disclosure is intended to cover various modifications and equivalent arrangements included within the spirit and scope of the appended claims.

All publications, patents and patent applications cited in the present disclosure are herein incorporated by reference in their entirety to the same extent as if each individual publication, patent or patent application was specifically and individually indicated to be incorporated by reference in its entirety.

### REFERENCES

1. Steim, R.; Ameri, T.; Schilinsky, P.; Waldauf, C.; Dennler, G.; Scharber, M.; Brabec, C. J. Organic Photovoltaics for Low Light Applications. Sol. Energy Mater. Sol. Cells 2011, 95 (12), 3256-3261.
2. Chen, T. W.; Karapala, V. K.; Chen, J. T.; Hsu, C. S. Recent Advances of Carbazole-Based Nonfullerene Acceptors: Molecular Design, Optoelectronic Properties, and Photovoltaic Performance in Organic Solar Cells. J. Chinese Chem. Soc. 2021, 68 (7), 1186-1196.
3. Harvey, P. D.; Sharma, G. D.; Witulski, B. Indolo- And Diindolocarbazoles in Organic Photovoltaic Cells. Chem. Lett. 2021, 50 (7), 1345-1355.
4. Karki, A.; Gillett, A. J.; Friend, R. H.; Nguyen, T. Q. The Path to 20% Power Conversion Efficiencies in Nonfullerene Acceptor Organic Solar Cells. Adv. Energy Mater. 2021, 11 (15), 2003441.
5. Chen, H.; Xia, X.; Yuan, J.; Wei, Q.; Liu, W.; Li, Z.; Zhu, C.; Wang, X.; Guan, H.; Lu, X.; Li, Y.; Zou, Y. Compatibility between Solubility and Enhanced Crystallinity of Benzotriazole-Based Small Molecular Acceptors with Less Bulky Alkyl Chains for Organic Solar Cells. ACS Appl. Mater. Interfaces 2021, 13 (30), 36053-36061.
6. Xiong, M.; Wu, J.; Fan, Q.; Liu, Q.; Lv, J.; Ou, X.; Guo, X.; Zhang, M. Ternary Organic Solar Cells with Improved Efficiency and Stability Enabled by Compatible Dual-Acceptor Strategy. Org. Electron. 2021, 96, 106227.
7. Xie, L.; Zhang, Y.; Zhuang, W.; Jeong, S. Y.; Bian, Q.; Li, H.; Cao, J.; Liu, W.; Tan, H.; Woo, H. Y.; Zhang, J.; Wang, E. Low-Bandgap Nonfullerene Acceptor Based on Thieno[3,2-b]Indole Core for Highly Efficient Binary and Ternary Organic Solar Cells. Chem. Eng. J. 2022, 427, 131674.
8. Gao, W.; Fu, H.; Li, Y.; Lin, F.; Sun, R.; Wu, Z.; Wu, X.; Zhong, C.; Min, J.; Luo, J.; Woo, H. Y.; Zhu, Z.; Jen, A. K. Y. Asymmetric Acceptors Enabling Organic Solar Cells to Achieve an over 17% Efficiency: Conformation Effects on Regulating Molecular Properties and Suppressing Nonradiative Energy Loss. Adv. Energy Mater. 2021, 11 (4), 2003177.
9. Zhang, Y.; Liu, K.; Huang, J.; Xia, X.; Cao, J.; Zhao, G.; Fong, P. W. K.; Zhu, Y.; Yan, F.; Yang, Y.; Lu, X.; Li, G. Graded Bulk-Heterojunction Enables 17% Binary Organic Solar Cells via Nonhalogenated Open Air Coating. Nat. Commun. 2021, 12 (1), 1-13.
10. Fan, H.; Yang, H.; Wu, Y.; Yildiz, O.; Zhu, X.; Marszalek, T.; Blom, P. W. M.; Cui, C.; Li, Y. Anthracene-Assisted Morphology Optimization in Photoactive Layer for High-Efficiency Polymer Solar Cells. Adv. Funct. Mater. 2021, 31 (37), 2103944.
11. Liu, Q.; Jiang, Y.; Jin, K.; Qin, J.; Xu, J.; Li, W.; Xiong, J.; Liu, J.; Xiao, Z.; Sun, K.; Yang, S.; Zhang, X.; Ding, L. 18% Efficiency Organic Solar Cells. Sci. Bull. 2020, 65 (4), 272-275.
12. Ma, L.; Zhang, S.; Wang, J.; Ren, J.; Gao, M.; Zhang, J.; Zhang, T.; Yao, H.; Ye, L.; Hou, J. Miscibility Control by Tuning Electrostatic Interactions in Bulk Heterojunction for Efficient Organic Solar Cells. ACS Mater. Lett. 2021, 12, 1276-1283.
13. Liu, F.; Zhou, L.; Liu, W.; Zhou, Z.; Yue, Q.; Zheng, W.; Sun, R.; Liu, W.; Xu, S.; Fan, H.; Feng, L.; Yi, Y.; Zhang, W.; Zhu, X. Organic Solar Cells with 18% Efficiency Enabled by an Alloy Acceptor: A Two-in-One Strategy. Adv. Mater. 2021, 33 (27), 2100830.
14. Zhan, L.; Li, S.; Xia, X.; Li, Y.; Lu, X.; Zuo, L.; Shi, M.; Chen, H. Layer-by-Layer Processed Ternary Organic Photovoltaics with Efficiency over 18%. Adv. Mater. 2021, 33 (12), 2007231.
15. Chen, S.; Feng, L.; Jia, T.; Jing, J.; Hu, Z.; Zhang, K.; Huang, F. High-Performance Polymer Solar Cells with Efficiency over 18% Enabled by Asymmetric Side Chain Engineering of Non-Fullerene Acceptors. Sci. China Chem. 2021, 64 (7), 1192-1199.
16. Su, Y. J.; Huang, S. C.; Chen, T. W.; Chueh, L. C.; Cui, Y.; Hong, L.; Yao, H.; Hou, J.; Chen, J. T.; Hsu, C. S. Elucidating End-Group Modifications of Carbazole-Based Nonfullerene Acceptors in Indoor Applications for Achieving a PCE of over 20%. ACS Appl. Mater. Interfaces 2021, 13 (22), 26247-26255.
17. Bucher, L.; Desbois, N.; Harvey, P. D.; Gros, C. P.; Misra, R.; Sharma, G. D. Nonfullerene Polymer Solar Cells Reaching a 9.29% Efficiency Using a BODIPY-Thiophene Backboned Donor Material. ACS Appl. Energy Mater. 2018, 1 (7), 3359-3368.
18. Rao, P. S.; More, V. G.; Jangale, A. D.; Bhosale, S. V.; Bhosale, R. S.; Puyad, A. L.; Chen, J. Y.; Li, J. L.; Bhosale, S. V.; Gupta, A.; Sharma, G. D. A Series of V-Shaped Small Molecule Non-Fullerene Electron Acceptors for Efficient Bulk-Heterojunction Devices. Dye. Pigment. 2019, 171, 107677.
19. Kono, T.; Shibata, Y.; Wang, Z.; Miyadera, T.; Yoshida, Y. Synthesis of Novel Push-Pull Chromophores Based on N-Ethylcarbazole for Vacuum Deposition Processed Organic Photovoltaics. Chem. Lett. 2015, 44 (7), 958-960.
20. Meshulam, G.; Berkovic, G.; Kotler, Z.; Ben-Asuly, A.; Mazor, R.; Shapiro, L.; Khodorkovsky, V. Effect of Carbazole as a Donor Moiety on the Second-Order Nonlinearity of Organic Molecules. In Organic Nonlinear Optical Materials; Eich, M., Kuzyk, M. G., Eds.; SPIE, 1999; Vol. 3796, pp 279-286.
21. Nowak-Król, A.; Wagener, R.; Kraus, F.; Mishra, A.; Bäuerle, P.; Würthner, F. Modulation of Band Gap and P-: Versus n-Semiconductor Character of ADA Dyes by Core and Acceptor Group Variation. Org. Chem. Front. 2016, 3 (5), 545-555.

1. A compound of Formula Ia: 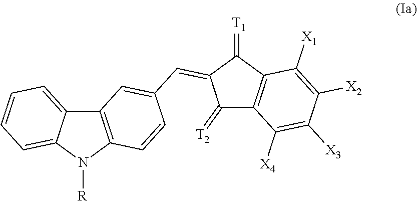
wherein:
R is a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

2. The compound of claim 1, comprising a structure of Formula Ila: 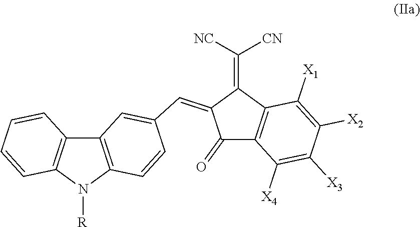
wherein:
R is a linear or branched C₆₋₁₅-alkyl; and
X₁, X₂, X₃ and X₄ are each independently H, F, Cl or Br.

3. The compound of claim 1, comprising a structure of Formula Illa: 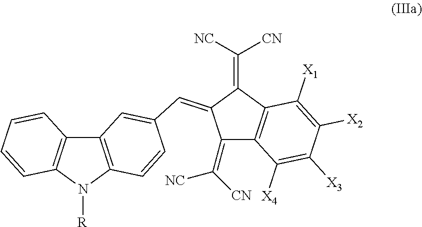
wherein:
R is a linear or branched C₆₋₁₅-alkyl; and
X₁, X₂, X₃ and X₄ are each independently H, F, Cl or Br.

4. The compound of claim 1, comprising a structure of Formula IVa: 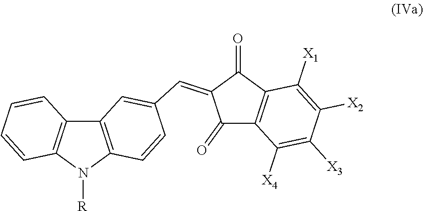
wherein:
R is a linear or branched C₆₋₁₅-alkyl; and
X₁, X₂, X₃ and X₄ are each independently H, F, Cl or Br.

5. The compound of any one of claims 2 to 4, wherein X₁, X₂, X₃ and X₄ are each independently H.

6. The compound of claim 5, wherein R is 2-ethylhexyl.

7. A compound of Formula Ib: 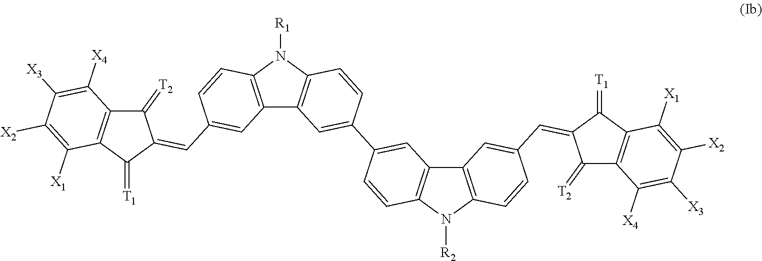
wherein:
R₁ and R₂ are each independently a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

8. The compound of claim 7, comprising a structure of Formula IIb: 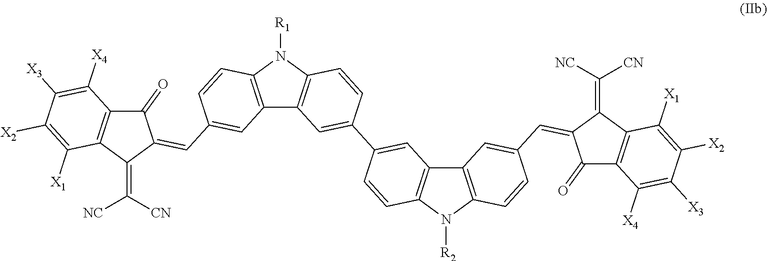
wherein:
R₁ and R₂ are each independently a linear or branched C₆₋₁₅-alkyl; and
X₁, X₂, X₃ and X₄ are each independently H, F, Cl or Br.

9. The compound of claim 8, comprising a structure of Formula IIIb: 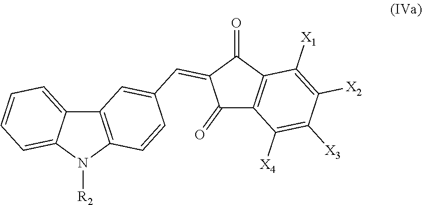
wherein:
R₁ and R₂ are each independently a linear or branched C₆₋₁₅-alkyl.

10. The compound of claim 8, comprising a structure of Formula IVb: 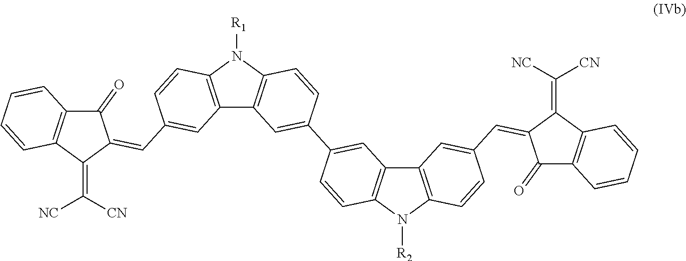
wherein:
R₁ and R₂ are each independently a linear or branched C₆₋₁₅-alkyl.

11. The compound of any one of claims 7 to 10, wherein R is 2-ethylhexyl.

12. A composition comprising an electron acceptor material A₁ and an electron donor material D₁, wherein the electron acceptor material A₁ is of Formula Ia: 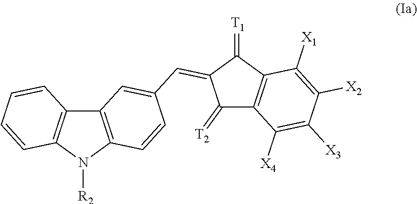
wherein:
R is a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

13. The composition of claim 12, wherein the electron acceptor material A₁ has the structure of Formula Ila: 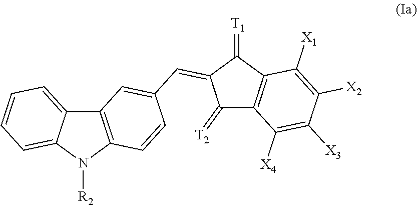
wherein:
R is a linear or branched C₆₋₁₅-alkyl; and
X₁, X₂, X₃ and X₄ are each independently H, F, Cl or Br.

14. The composition of claim 12, wherein the electron acceptor material A₁ has the structure of Formula IIIa: 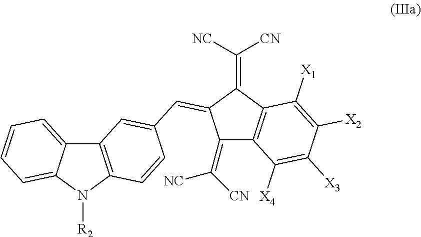
wherein:
R is a linear or branched C₆₋₁₅-alkyl; and
X₁, X₂, X₃ and X₄ are each independently H, F, Cl or Br.

15. The composition of claim 12, wherein the electron acceptor material A₁ has the structure of Formula IVa: 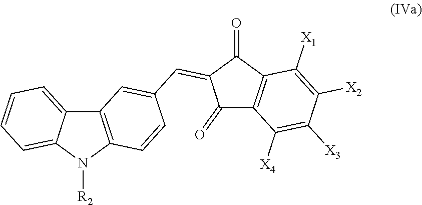
wherein:
R is a linear or branched C₆₋₁₅-alkyl; and
X₁, X₂, X₃ and X₄ are each independently H, F, Cl or Br.

16. The composition of any one of claims 12 to 15, wherein X₁, X₂, X₃ and X₄ are each independently H.

17. The composition of claim 16, wherein R is 2-ethylhexyl.

18. A composition comprising an electron acceptor material A₁ and an electron donor material D₁, wherein the electron acceptor material A₁ is of Formula Ib: 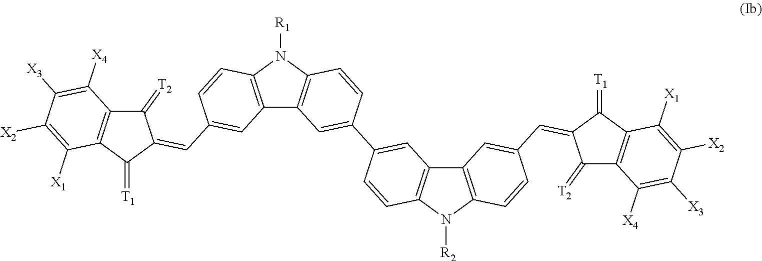
wherein:
R₁ and R₂ are each independently a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

19. The composition of claim 18, wherein the electron acceptor material A₁ has the structure of Formula IIb: 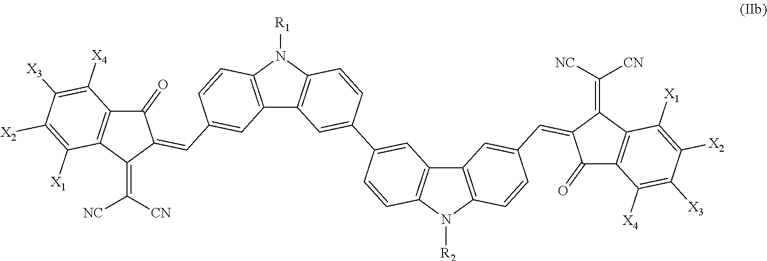
wherein:
R₁ and R₂ are each independently a linear or branched C₆₋₁₅-alkyl; and
X₁, X₂, X₃ and X₄ are each independently H, F, Cl or Br.

20. The composition of claim 18, wherein the electron acceptor material A₁ has the structure of Formula IIIb: 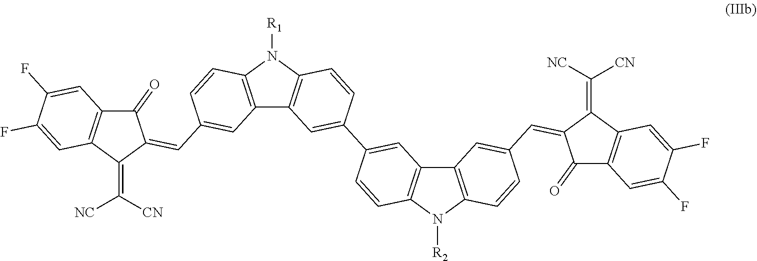
wherein:
R₁ and R₂ are each independently a linear or branched C₆₋₁₅-alkyl.

21. The composition of claim 18, wherein the electron acceptor material A₁ has the structure of Formula IVb: 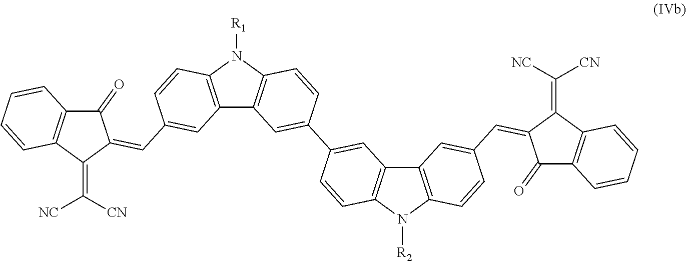
wherein:
R₁ and R₂ are each independently a linear or branched C₆₋₁₅-alkyl.

22. The composition of any one of claims 18 to 21, wherein R is 2-ethylhexyl.

23. The composition of any one of claims 12 to 22, wherein the electron donor material D₁ comprises a p-type organic semiconductor material.

24. The composition of any one of claims 12 to 23, wherein the electron donor material D₁ comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT).

25. The composition of any one of claims 12 or 24, wherein the composition comprises a mass ratio (w/w) of acceptor material to donor material ranging from about 0.5:1.0 to about 1.0:0.5.

26. The composition of any one of claims 12 to 25, wherein the composition is slot die coated.

27. The composition of any one of claims 12 to 25, wherein the composition is spin coated.

28. The composition of any one of claims 12 to 27, wherein the composition produces a band gap suitable for low intensity light harvesting, and wherein the blend has significant absorption of visible light between 380 nm and 940 nm suitable for low intensity light harvesting.

29. The composition of any one of claims 12 to 28, wherein the composition is provided in the form of a bulk material or a film.

30. A composition comprising at least two electron acceptor materials A₁ and A₂, and at least an electron donor material D₁, wherein the electron acceptor materials A₁ and A₂, are of Formula Ia: 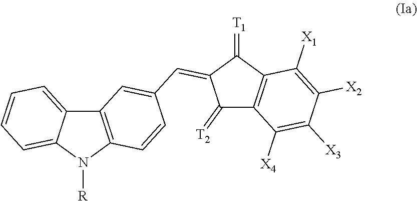
wherein:
R is a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

31. The composition of claim 30, wherein at least one of the two electron acceptor materials A₁ and A₂ has the structure of Formula Ila: 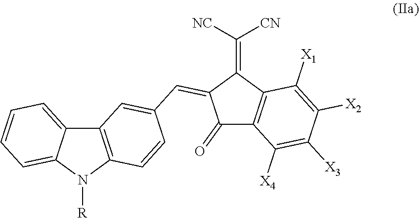
wherein:
R is a linear or branched C₆₋₁₅-alkyl; and
X₁, X₂, X₃ and X₄ are each independently H, F, Cl or Br.

32. The composition of claim 30, wherein at least one of the two electron acceptor materials A₁ and A₂ has the structure of Formula IIIa: 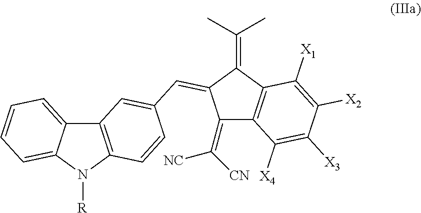
wherein:
R is a linear or branched C₆₋₁₅-alkyl; and
X₁, X₂, X₃ and X₄ are each independently H, F, Cl or Br.

33. The composition of claim 30, wherein at least one of the two electron acceptor materials A₁ and A₂ has the structure of Formula IVa: 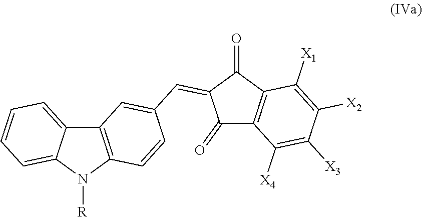
wherein:
R is a linear or branched C₆₋₁₅-alkyl; and
X₁, X₂, X₃ and X₄ are each independently H, F, Cl or Br.

34. The composition of any one of claims 30 to 33, wherein X₁, X₂, X₃ and X₄ are each independently H.

35. The composition of claim 34, wherein R is 2-ethylhexyl.

36. A composition comprising at least two electron acceptor materials A₁ and A₂, and at least an electron donor material D₁, wherein the electron acceptor materials A₁ and A₂, are of Formula Ib: 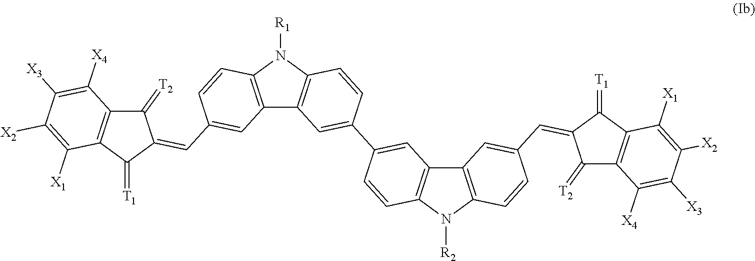
wherein:
R₁ and R₂ are each independently a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

37. The composition of claim 36, wherein at least one of the two electron acceptor materials A₁ and A₂ has the structure of Formula IIb: 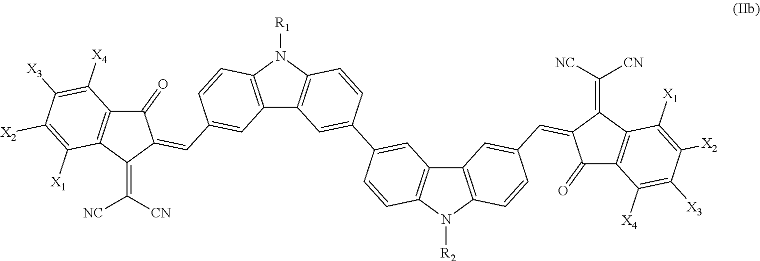
wherein:
R₁ and R₂ are each independently a linear or branched C₆₋₁₅-alkyl; and
X₁, X₂, X₃ and X₄ are each independently H, F, Cl or Br.

38. The composition of claim 36, wherein at least one of the two electron acceptor materials A₁ and A₂ has the structure of Formula IIIb: 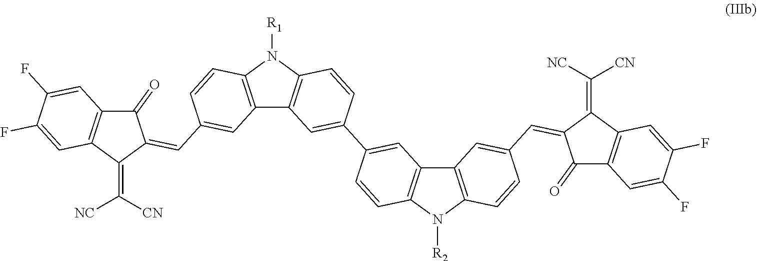
wherein:
R₁ and R₂ are each independently a linear or branched C₆₋₁₅-alkyl.

39. The composition of claim 36, wherein at least one of the two electron acceptor materials A₁ and A₂ has the structure of Formula IVb: 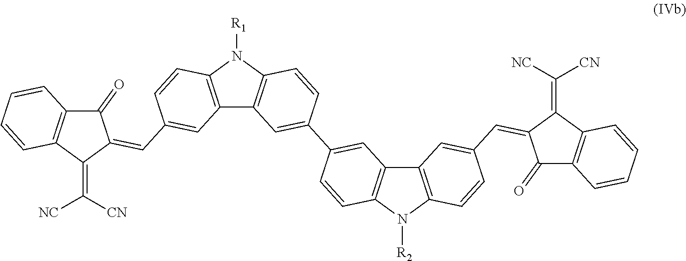
wherein:
R₁ and R₂ are each independently a linear or branched C₆₋₁₅-alkyl.

40. The composition of any one of claims 36 to 39, wherein R is 2-ethylhexyl.

41. The composition of any one of claims 30 to 40, wherein the electron donor material D₁ comprises a p-type organic semiconductor material.

42. The composition of any one of claims 30 to 41, wherein the electron donor material D₁ comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT).

43. The composition of any one of claims 30 to 42, wherein the composition comprises a mass ratio (w/w) of acceptor material to donor material ranging from about 0.5:1.0 to about 1.0:0.5.

44. The composition of any one of claims 30 to 43, wherein the composition is slot die coated.

45. The composition of any one of claims 30 to 43, wherein the composition is spin coated.

46. The composition of any one of claims 30 to 45, wherein the composition produces a band gap suitable for low intensity light harvesting, and wherein the blend has significant absorption of visible light between 380 nm and 940 nm suitable for low intensity light harvesting.

47. The composition of any one of claims 30 to 46, wherein the composition is provided in the form of a bulk material or a film.

48. A composition comprising at least two electron acceptor materials A₁ and A₂, and at least an electron donor material D₁, wherein the electron acceptor materials A₁ and A₂, are of Formula Ia and/or Ib: 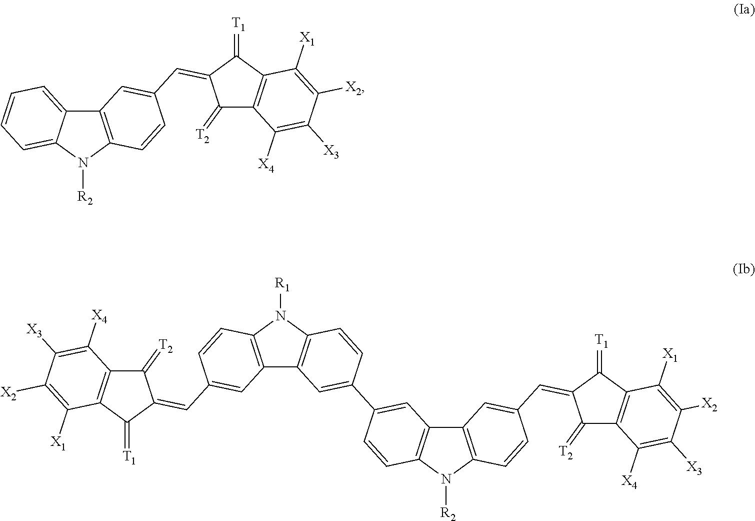
wherein:
R, R₁ and R₂ are each independently a linear or branched C₆₋₁₅-alkyl;
X₁, X₂, X₃ and X₄ are each independently H, F, Cl or Br; and
T₁ and T₂ are each independently ═O, ═C(CN)₂, or ═CHCN.

49. The composition of claim 48, wherein at least one of the two electron acceptor materials A₁ and A₂ has the structure of Formula Ila: 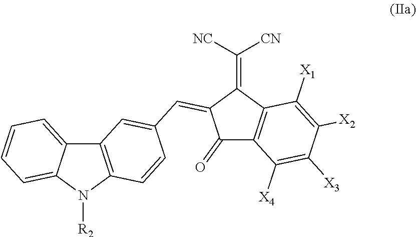
wherein:
R is a linear or branched C₆₋₁₅-alkyl; and
X₁, X₂, X₃ and X₄ are each independently H, F, Cl or Br.

50. The composition of claim 48, wherein at least one of the two electron acceptor materials A₁ and A₂ has the structure of Formula IIIa: 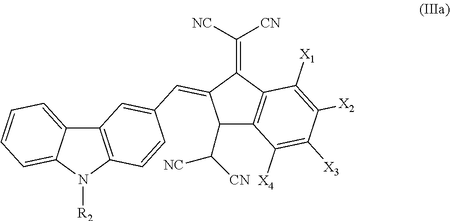
wherein:
R is a linear or branched C₆₋₁₅-alkyl; and
X₁, X₂, X₃ and X₄ are each independently H, F, Cl or Br.

51. The composition of claim 48, wherein at least one of the two electron acceptor materials A₁ and A₂ has the structure of Formula IVa: 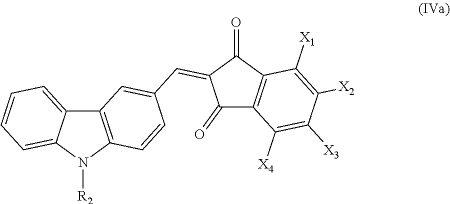
wherein:
R is a linear or branched C₆₋₁₅-alkyl; and
X₁, X₂, X₃ and X₄ are each independently H, F, Cl or Br.

52. The composition of claim 48, wherein at least one of the two electron acceptor materials A₁ and A₂ has the structure of Formula IIb: 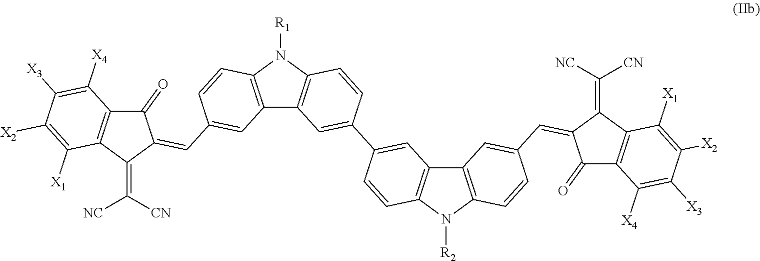
wherein:
R₁ and R₂ are each independently a linear or branched C₆₋₁₅-alkyl; and
X₁, X₂, X₃ and X₄ are each independently H, F, Cl or Br.

53. The composition of claim 48, wherein at least one of the two electron acceptor materials A₁ and A₂ has the structure of Formula IIIb: 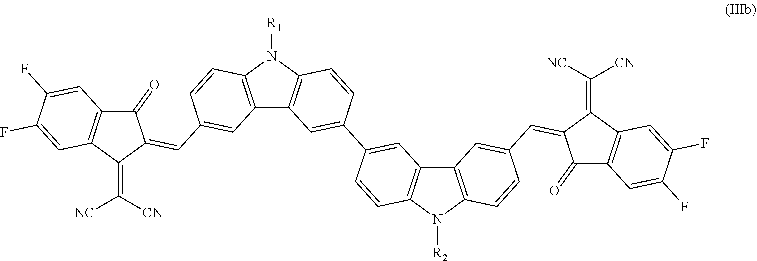
wherein:
R₁ and R₂ are each independently a linear or branched C₆₋₁₅-alkyl.

54. The composition of claim 48, wherein at least one of the two electron acceptor materials A₁ and A₂ has the structure of Formula IVb: 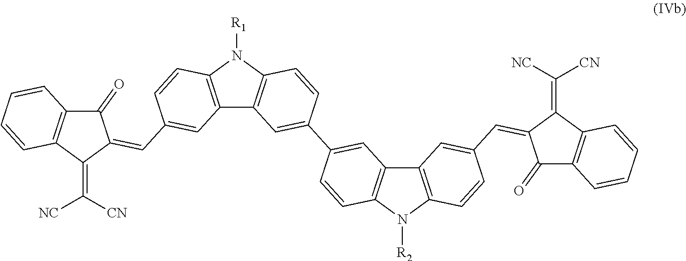
wherein:
R₁ and R₂ are each independently a linear or branched C₆₋₁₅-alkyl.

55. The composition of any one of claims 48 to 54, wherein the electron donor material D₁ comprises a p-type organic semiconductor material.

56. The composition of any one of claims 48 to 55, wherein the electron donor material D₁ comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT).

57. The composition of any one of claims 48 to 56, wherein the composition comprises a mass ratio (w/w) of acceptor material to donor material ranging from about 0.5:1.0 to about 1.0:0.5.

58. The composition of any one of claims 48 to 57, wherein the composition is slot die coated.

59. The composition of any one of claims 48 to 57, wherein the composition is spin coated.

60. The composition of any one of claims 48 to 59, wherein the composition produces a band gap suitable for low intensity light harvesting, and wherein the blend has significant absorption of visible light between 380 nm and 940 nm suitable for low intensity light harvesting.

61. The composition of any one of claims 48 to 60, wherein the composition is provided in the form of a bulk material or a film.

62. Use of a compound as defined in any one of claims 1 to 11 as an electron acceptor compound.

63. An organic solar cell, comprising:
an anode and a cathode; and
a photoactive layer between the anode and the cathode;
wherein the photoactive layer comprises an electron donor/acceptor material,
wherein the electron acceptor material is as defined in any one of claims 1 to 11.

64. The organic solar cell of claim 63, wherein the electron donor material comprises a p-type organic semiconductor material.

65. The organic solar cell of claim 63 or 64, wherein the electron donor material comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT).

66. The organic solar cell of any one of claims 63 to 65, wherein the photoactive layer comprises a mass ratio (w/w) of acceptor material to donor material ranging from about 0.5:1.0 to about 1.0:0.5.

67. The organic solar cell of any one of claims 63 to 66, wherein the photoactive layer is slot die coated.

68. The organic solar cell of any one of claims 63 to 66, wherein the photoactive layer is spin coated.

69. The organic solar cell of any one of claims 63 to 68, wherein one of the cathode and the anode comprises one of indium tin oxide (ITO), indium-doped zinc oxide (IZO), tin oxide (SnO), aluminum doped zinc oxide (AZO), or gallium-doped zinc oxide (GZO), and the other of the cathode and the anode includes one of aluminum (Al), silver (Ag), gold (Au), or lithium (Li).

70. The organic solar cell of any one of claims 63 to 69, wherein the photoactive layer produces a band gap suitable for low intensity light harvesting, and wherein the photoactive layer has significant absorption of visible light between 380 nm and 940 nm suitable for low intensity light harvesting.

71. The organic solar cell of any one of claims 63 to 70, wherein the photoactive layer is provided in the form of a bulk material or a film.

72. An electronic device comprising a heterojunction, wherein the heterojunction comprises a blend comprising an electron donor and an electron acceptor material, wherein the electron acceptor material is as defined in any one of claims 1 to 11.

73. The electronic device of claim 72, wherein the electron donor material comprises a p-type organic semiconductor material.

74. The electronic device of claim 72 or 73, wherein the electron donor material comprises (poly([2,6′-4,8-di(5-ethylhexylthienyl)benzo[1,2-b;3,3-b]dithiophene]{3-fluoro-2[(2-ethylhexyl)carbonyl]thieno[3,4-b]thiophenediyl})) (PTB7-Th), poly(3-hexylthiophene) (P3HT), poly[(2,5-bis(2-hexyldecyloxy) phenylene)-alt-(5,6-difluoro-4,7-di(thiophen-2-yl)benzo[c][1,2,5]thiadiazole)](PPDT2FBT) or poly[9-(1-octylnonyl)-9H-carbazole-2,7-diyl]-2,5-thiophenediyl-2,1,3-benzothiadiazole-4,7-diyl-2,5-thiophenediyl](PCDTBT).

75. The electronic device of any one of claims 72 to 74, wherein the heterojunction comprises a mass ratio (w/w) of acceptor material to donor material ranging from about 0.5:1.0 to about 1.0:0.5.

76. The electronic device of any one of claims 72 to 75, wherein the blend is slot die coated.

77. The electronic device of any one of claims 72 to 75, wherein the blend is spin coated.

78. The electronic device of any one of claims 72 to 77, wherein the blend produces a band gap suitable for low intensity light harvesting, and wherein the blend has significant absorption of visible light between 380 nm and 940 nm suitable for low intensity light harvesting.

79. The electronic device of any one of claims 72 to 78, wherein the blend is provided in the form of a bulk material or a film.

80. The electronic device of claim 72, wherein the device is a photovoltaic cell, an organic transistor, a light emitting diode, or a photodetector.

81. An organic semiconductor material, layer or component, comprising the composition as defined in any one of claims 12 to 29.

82. An organic semiconductor material, layer or component, comprising the composition as defined in any one of claims 30 to 47.

83. An organic semiconductor material, layer or component, comprising the composition as defined in any one of claims 48 to 61.

84. Use of the composition as defined in any one of claims 12 to 29 as an organic semiconductor material, layer or component.

85. Use of the composition as defined in any one of claims 30 to 47 as an organic semiconductor material, layer or component.

86. Use of the composition as defined in any one of claims 48 to 61 as an organic semiconductor material, layer or component.

87. Use of the composition as defined in any one of claims 12 to 29 in an electronic device.

88. Use of the composition as defined in any one of claims 30 to 47 in an electronic device.

89. Use of the composition as defined in any one of claims 48 to 61 in an electronic device.

90. The use of any one of claims 84 to 86, wherein the electronic device is a photovoltaic cell, an organic transistor, a light emitting diode, or a photodetector.

91. A bulk heterojunction (BHJ) formed from a composition as defined in any one of claims 12 to 29.

92. A bulk heterojunction (BHJ) formed from a composition as defined in any one of claims 30 to 47.

93. A bulk heterojunction (BHJ) formed from a composition as defined in any one of claims 48 to 61.
